(19) United States
(12) Patent Application Publication
(10) Pub. No.: US 2022/0204610 A1
(43) Pub. Date: Jun. 30, 2022

(54) PROTEIN BINDERS FOR IRHOM2

(71) Applicants: NEW YORK SOCIETY FOR THE RELIEF OF THE RUPTURED AND CRIPPLED, MAINTAING HOSPITAL FOR SPECIAL SURGERY, New York, NY (US); SCIRHOM GMBH, Planegg (DE)

(72) Inventors: Matthias Schneider, Neufarn (DE); Kerstin Selle, Planegg (DE); Jens Ruhe, Planegg (DE); Gisela Weskamp, Eastchester, NY (US); Carl Blobel, Eastchester, NY (US)

(21) Appl. No.: 17/602,183

(22) PCT Filed: Apr. 9, 2020
(86) PCT No.: PCT/EP2020/060179

§ 371 Date: Oct. 7, 2021

(30) Foreign Application Priority Data
Apr. 9, 2019 (EP) 19168218.6
Nov. 15, 2019 (EP) 19209426.6

(51) Int. Cl. C07K 16/28

(52) U.S. Cl. CPC C07K 16/28; C07K 2317/33; C07K 2317/92; C07K 2317/76; C07K 2317/34

(57) ABSTRACT

The present invention relates to a protein binder that binds to human iRhom2, and inhibits and/or reduces TACE/ADAM17 activity when bound to human iRhom2.

### FIELD OF THE INVENTION

The present application relates to Protein binders for iRhom2.

### BACKGROUND

ADAM metallopeptidase domain 17 (ADAM17) (NCBI reference of human ADAM17: NP 003174), also called TACE (tumor necrosis factor-α-converting enzyme), is a 70-kDa enzyme that belongs to the ADAM protein family of disintegrins and metalloproteases. It is an 824-amino acid polypeptide.

ADAM17 is understood to be involved in the processing of tumor necrosis factor alpha (TNF-α) at the surface of the cell, and from within the intracellular membranes of the trans-Golgi network. This process, which is also known as ‘shedding’, involves the cleavage and release of a soluble ectodomain from membrane-bound pro-proteins (such as pro-TNF-α), and is of known physiological importance. ADAM17 was the first ‘sheddase’ to be identified, and is also understood to play a role in the release of a diverse variety of membrane-anchored cytokines, cell adhesion molecules, receptors, ligands, and enzymes.

Cloning of the TNF-α gene revealed it to encode a 26 kDa type II transmembrane pro-polypeptide that becomes inserted into the cell membrane during its translocation in the endoplasmic reticulum. At the cell surface, pro-TNF-α is biologically active, and is able to induce immune responses via juxtacrine intercellular signaling. However, pro-TNF-α can undergo proteolytic cleavage at its Ala76-Va177 amide bond, which releases a soluble 17 kDa extracellular domain (ectodomain) from the pro-TNF-α molecule. This soluble ectodomain is the cytokine commonly known as TNF-α, which is of pivotal importance in paracrine signaling of this molecule. This proteolytic liberation of soluble TNF-α is catalyzed by ADAM17.

ADAM17 also modulates the MAP kinase signaling pathway by regulating the cleavage of the EGFR ligand amphiregulin in the mammary gland. Moreover, ADAM17 has a role in shedding of L-selectin, a cellular adhesion molecule.

Recently, ADAM17 was discovered as a crucial mediator of resistance formation to radiotherapy. Radiotherapy can induce a dose-dependent increase of furin-mediated cleavage of the ADAM17 proform to active ADAM17, which results in enhanced ADAM17 activity in vitro and in vivo. It was also shown that radiotherapy activates ADAM17 in non-small cell lung cancer, which results in shedding of multiple survival factors, growth factor pathway activation, and radiotherapy-induced treatment resistance.

Since ADAM17 seems to be a crucial factor for the release of different pathogenic and non-pathogenic factors, including TNFα, it has come into the focus as therapeutic target molecule. For that reason, different attempts have been made to develop inhibitors of ADAM17.

However, so far, no such inhibitor has proven clinically successful.

It is hence one object of the present invention to provide a new approach which allows the control, regulation, reduction or inhibition of ADAM17 activity.

It is another object of the present invention to provide a new approach that allows the treatment of inflammatory diseases.

These and other objects are solved by the features of the independent claims. The dependent claims disclose embodiments of the invention which may be preferred under particular circumstances. Likewise, the specification discloses further embodiments of the invention which may be preferred under particular circumstances.

### SUMMARY OF THE INVENTION

The present invention provides, among others, a protein binder that binds to human iRhom2, and inhibits and/or reduces TACE/ADAM17 activity when bound to human iRhom2.

### BRIEF DESCRIPTION OF THE DRAWINGS

FIG. 1 shows the sequences of the peptides used herein for immunization and peptide binding ELISA analyses. These peptides are subsequences of the entire iRhom2 or iRhom1 sequence. To increase immunogenicity, some peptides have been conjugated with KLH (keyhole limpet hemocyanin) via the SH-group of a cysteine. For peptide binding analysis, these peptides have been conjugated to Biotin instead. For that purpose, either a cysteine was used, which naturally occurred on either the N- or C-terminus of the respective peptide, or a cysteine was added to either N- or C-terminus (marked as “—C—” in FIG. 1). To avoid unspecific intrachain disulfide bond formation or unspecific intrachain conjugation of the KLH and/or Biotin, intrachain cysteines were replaced by aminobutyrate (marked as “Abu” in FIG. 1).

FIG. 2 shows results from TNFα release assays (shedding assays) for functional screening of hybridoma supernatants, demonstrating that the supernatant of the hybridoma clone 4H8 effectively interferes with LPS-induced shedding of TNFα in THP-1 cells.

FIG. 3 depicts results from ELISA analyses for antibody isotype determination demonstrating the antibody 4H8-E3 of the invention to be of mouse IgM isotype.

FIG. 4 shows results from peptide binding ELISA analyses revealing the antibody 4H8-E3 of the invention to recognize an epitope within the section of the large extracellular loop 1 of human iRhom2 (“juxtamembrane domain”, JMD) that is adjacent to the 1st “transmembrane domain” (TMD1).

The antibody 4H8-E3 of the invention recognizes peptide 3, which corresponds to amino acids 431 to 459 of human iRhom2, which is the JMD section of the large extracellular loop 1 of human iRhom2 (“juxtamembrane domain”) adjacent to TMD1.

FIG. 5 shows results from peptide binding ELISA analyses revealing the antibody 4H8-E3 of the invention to recognize an epitope within the extracellular juxtamembrane region adjacent to the TMD1 of human iRhom2, but not within the homologous region of human iRhom1. The antibody 4H8-E3 of the invention recognizes peptide 3, but not peptide 3b, which corresponds to the respective homologous section of human iRhom1.

FIG. 6 shows results from TNFα release assays demonstrating the antibody 4H8-E3 of the invention to inhibit LPS-induced shedding of TNFα in THP-1 cells.

FIG. 7 shows results from TNFα release assays demonstrating the concentration-dependent inhibition of LPS-induced TNFα shedding by the antibody 4H8-E3 of the invention in THP-1 cells.

FIG. 8 shows a schematic representation of iRhom2 with the positions of the juxtamembrane domain adjacent to the TMD1 (A), loop 1 (B) and the C-terminus (C) being illustrated.

FIG. 9 depicts the amino acid sequence of human iRhom2 according to SEQ ID NO 16, with the sequences shown which correspond to the immunization peptides used in this invention.

FIG. 10 shows an alignment of human iRhom2 according to SEQ ID NO 16 and human iRhom1 according to SEQ ID NO 17. The grey area shows sequence which corresponds to immunization peptide 3 used in this invention.

### DETAILED DESCRIPTION

According to one aspect of the invention, a protein binder is provided that binds to human iRhom2, and inhibits and/or reduces TACE/ADAM17 activity when bound to human iRhom2.

Rhomboid family member 2 (iRhom2) is a protein that in humans is encoded by the RHBDF2 gene. It is a transmembrane protein consisting of about 850 amino acids, having seven transmembrane domains. The inventors of the present invention have for the first time demonstrated that iRhom2 can act as a target for protein binders to inhibit TACE/ADAM17 activity.

iRhom2 comes in different isoforms. The experiments made herein have been established with the isoform defined as NCBI reference NP 078875.4. However, the teachings are transferable, without limitation, to other isoforms of iRhom2, as shown in the following table:

| mRNA | protein | name |
|---|---|---|
| NM_024599.5 | NP 078875.4 | inactive rhomboid protein 2 |
| | | transcript variant 1/isoform 1 |
| NM 001005498.3 | NP 001005498.2 | inactive rhomboid protein 2 |
| | | transcript variant 2/isoform 2 |

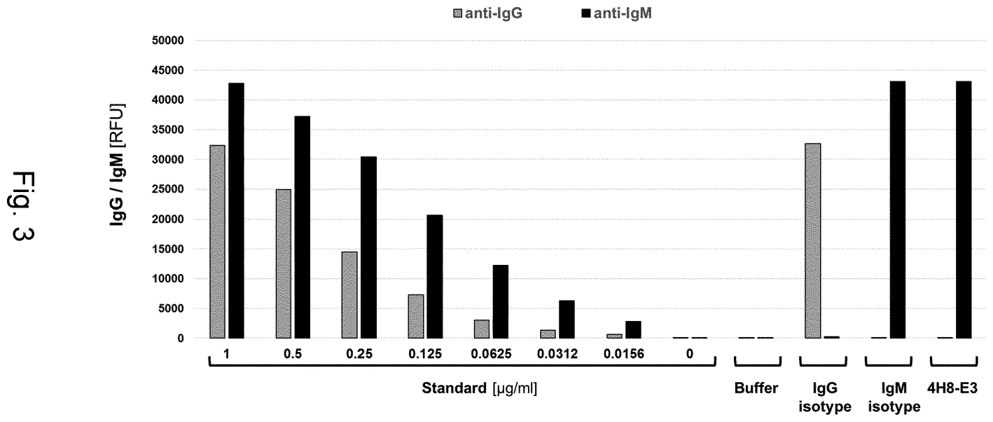

As used herein, the term “inhibits and/or reduces TACE/ADAM17 activity is meant to describe an effect caused by a protein binder that blocks or reduces the activity of TACE/ADAM17, as measured e.g. in a respective shedding assay (see., e.g., FIG. 2 and example 5).

According to one or more embodiments, the protein binder is a monoclonal antibody, or a target-binding fragment or derivative thereof retaining target binding capacities, or an antibody mimetic.

As used herein, the term “monoclonal antibody (mAb)” shall refer to an antibody composition having a homogenous antibody population, i.e., a homogeneous population consisting of a whole immunoglobulin, or a fragment or derivative thereof retaining target binding capacities. Particularly preferred, such antibody is selected from the group consisting of IgG, IgD, IgE, IgA and/or IgM, or a fragment or derivative thereof retaining target binding capacities.

As used herein, the term “fragment” shall refer to fragments of such antibody retaining target binding capacities, e.g.
a CDR (complementarity determining region)
a hypervariable region,
a variable domain (Fv)
an IgG or IgM heavy chain (consisting of VH, CH1 hinge, CH2 and CH3 regions)
an IgG or IgM light chain (consisting of VL and CL regions), and/or
a Fab and/or F(ab)₂.

As used herein, the term “derivative” shall refer to protein constructs being structurally different from, but still having some structural relationship to, the common antibody concept, e.g., scFv, Fab and/or F(ab)2, as well as bi-, tri- or higher specific antibody constructs, and further retaining target binding capacities. All these items are explained below.

Other antibody derivatives known to the skilled person are Diabodies, Camelid Antibodies, Nanobodies, Domain Antibodies, bivalent homodimers with two chains consisting of scFvs, IgAs (two IgG structures joined by a J chain and a secretory component), shark antibodies, antibodies consisting of new world primate framework plus non-new world primate CDR, dimerized constructs comprising CH3+VL+VH, and antibody conjugates (e.g. antibody or fragments or derivatives linked to a toxin, a cytokine, a radioisotope or a label). These types are well described in the literature and can be used by the skilled person on the basis of the present disclosure, without adding further inventive activity.

Methods for the production of a hybridoma cell are disclosed in Köhler & Milstein (1975).

Methods for the production and/or selection of chimeric or humanised mAbs are known in the art. For example, U.S. Pat. No. 6,331,415 by Genentech describes the production of chimeric antibodies, while U.S. Pat. No. 6,548,640 by Medical Research Council describes CDR grafting techniques and U.S. Pat. No. 5,859,205 by Celltech describes the production of humanised antibodies.

Methods for the production and/or selection of fully human mAbs are known in the art. These can involve the use of a transgenic animal which is immunized with the respective protein or peptide, or the use of a suitable display technique, like yeast display, phage display, B-cell display or ribosome display, where antibodies from a library are screened against human iRhom2 in a stationary phase.

In vitro antibody libraries are, among others, disclosed in U.S. Pat. No. 6,300,064 by MorphoSys and U.S. Pat. No. 6,248,516 by MRC/Scripps/Stratagene. Phage Display techniques are for example disclosed in U.S. Pat. No. 5,223,409 by Dyax. Transgenic mammal platforms are for example described in EP1480515A2 by TaconicArtemis.

IgG, IgM, scFv, Fab and/or F(ab)₂ are antibody formats well known to the skilled person. Related enabling techniques are available from the respective textbooks.

As used herein, the term “Fab” relates to an IgG/IgM fragment comprising the antigen binding region, said fragment being composed of one constant and one variable domain from each heavy and light chain of the antibody

As used herein, the term “F(ab)₂” relates to an IgG/IgM fragment consisting of two Fab fragments connected to one another by disulfide bonds.

As used herein, the term “scFv” relates to a single-chain variable fragment being a fusion of the variable regions of the heavy and light chains of immunoglobulins, linked together with a short linker, usually serine (S) or glycine (G). This chimeric molecule retains the specificity of the original immunoglobulin, despite removal of the constant regions and the introduction of a linker peptide.

Modified antibody formats are for example bi- or trispecific antibody constructs, antibody-based fusion proteins, immunoconjugates and the like. These types are well described in the literature and can be used by the skilled person on the basis of the present disclosure, with adding further inventive activity.

As used herein, the term “antibody mimetic” relates to an organic molecule, most often a protein that specifically binds to a target protein, similar to an antibody, but is not structurally related to antibodies. Antibody mimetics are usually artificial peptides or proteins with a molar mass of about 3 to 20 kDa. The definition encompasses, inter alia, Affibody molecules, Affilins, Affimers, Affitins, Alphabodies, Anticalins, Avimers, DARPins, Fynomers, Kunitz domain peptides, Monobodies, and nanoCLAMPs.

In one or more embodiments, the protein binder is an isolated antibody, or a target-binding fragment or derivative thereof retaining target binding capacities, or an isolated antibody mimetic

In one or more embodiments, the antibody is an engineered or recombinant antibody, or a target binding fragment or derivative thereof retaining target binding capacities, or an engineered or recombinant antibody mimetic.

According to one or more embodiments of the invention, the inhibition or reduction of TACE/ADAM17 activity is caused by interference with iRhom2-mediated TACE/ADAM17 activation.

According to one or more embodiments of the invention, the antibody inhibits or reduces TNFα shedding.

TNFα shedding, as used herein, refers to a process in which membrane-anchored tumor necrosis factor alpha (mTNFα/pro-TNFα) is released into the environment to become soluble TNFα (sTNFα or simply TNFα). This process is, inter alia, triggered by TACE/ADAM17.

According to one or more embodiments of the invention, the human iRhom2 to which the protein binder binds comprises
a) the amino acid sequence set forth in SEQ ID NO 16, or
b) an amino acid sequence that has at least 80% sequence identity with SEQ ID NO 16, with the proviso that said sequence maintains iRhom2 activity.

In some embodiments, human iRhom2 comprises an amino acid sequence that has ≥81%, preferably ≥82%, more preferably ≥83%, ≥84%, ≥85%, ≥86%, ≥87%, ≥88%, ≥89%, ≥90%, ≥91%, ≥92%, ≥93%, ≥94%, ≥95%, ≥96%, ≥97%, ≥98 or most preferably ≥99% sequence identity with SEQ ID NO 16.

SEQ ID NO 16 represents the amino acid sequence of inactive rhomboid protein 2 (iRhom2) isoform 1 [Homo sapiens], accessible under NCBI reference NP_078875.4. Generally, different variants and isoforms of iRhom2 exist. Likewise, mutants comprising conservative or silent amino acid substitutions exist, or may exist, which maintain full or at least substantial iRhom2 activity. These isoforms, variants and mutants are encompassed by the identity range specified above, meaning however that dysfunctional, non-active variants and mutants are excluded.

In this context, a “conservative amino acid substitution”, has a smaller effect on antibody function than a non-conservative substitution. Although there are many ways to classify amino acids, they are often sorted into six main groups on the basis of their structure and the general chemical characteristics of their R groups.

In some embodiments, a “conservative amino acid substitution” is one in which the amino acid residue is replaced with an amino acid residue having a similar side chain. For example, families of amino acid residues having similar side chains have been defined in the art. These families include amino acids with
basic side chains (e.g., lysine, arginine, histidine),
acidic side chains (e.g., aspartic acid, glutamic acid),
uncharged polar side chains (e.g., glycine, asparagine, glutamine, serine, threonine, tyrosine, cysteine),
nonpolar side chains (e.g., alanine, valine, leucine, isoleucine, proline, phenylalanine, methionine, tryptophan),
beta-branched side chains (e.g., threonine, valine, isoleucine) and
aromatic side chains (e.g., tyrosine, phenylalanine, tryptophan, histidine).

Other conserved amino acid substitutions can also occur across amino acid side chain families, such as when substituting an asparagine for aspartic acid in order to modify the charge of a peptide. Conservative changes can further include substitution of chemically homologous non-natural amino acids (i.e. a synthetic non-natural hydrophobic amino acid in place of leucine, a synthetic non-natural aromatic amino acid in place of tryptophan).

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (e.g., a polypeptide), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., at least 85%, 90%, 95%, 96%, 97%, 98% or 99% sequence identity over a specified region, or, when not specified, over the entire sequence of a reference sequence), when compared and aligned for maximum correspondence over a comparison window, or designated region as measured using one of the following sequence comparison algorithms or by manual alignment and visual inspection. The disclosure provides polypeptides or polynucleotides that are substantially identical to the polypeptides or polynucleotides, respectively, exemplified herein. Optionally, the identity exists over a region that is at least about 15, 25 or 50 nucleotides in length, or more preferably over a region that is 100 to 500 or 1000 or more nucleotides in length, or over the full length of the reference sequence. With respect to amino acid sequences, identity or substantial identity can exist over a region that is at least 5, 10, 15 or 20 amino acids in length, optionally at least about 25, 30, 35, 40, 50, 75 or 100 amino acids in length, optionally at least about 150, 200 or 250 amino acids in length, or over the full length of the reference sequence. With respect to shorter amino acid sequences, e.g., amino acid sequences of 20 or fewer amino acids, substantial identity exists when one or two amino acid residues are conservatively substituted, according to the conservative substitutions defined herein.

According to one or more embodiments of the invention, the protein binder binds to the extracellular juxtamembrane domain adjacent to the transmembrane domain 1 (TMD1) of human iRhom2.

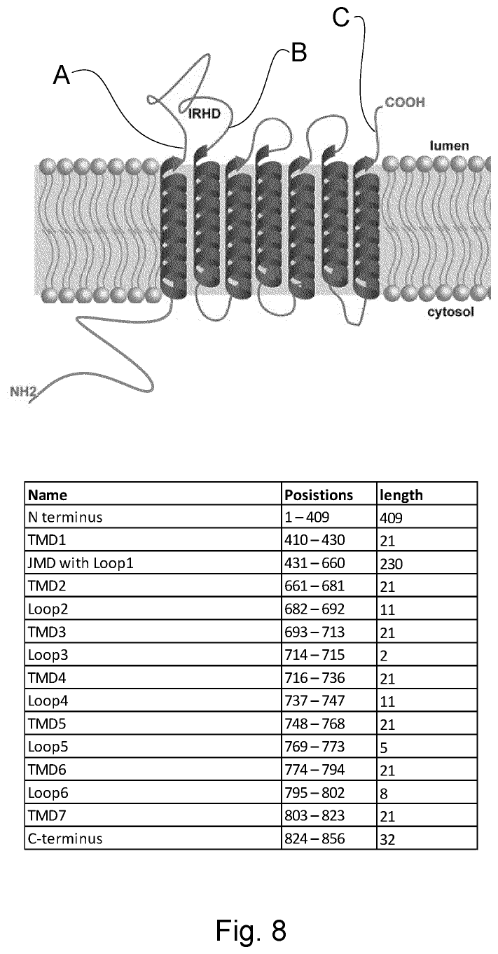

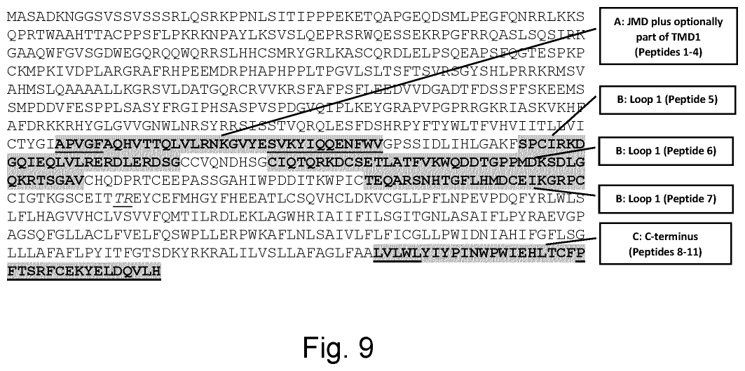

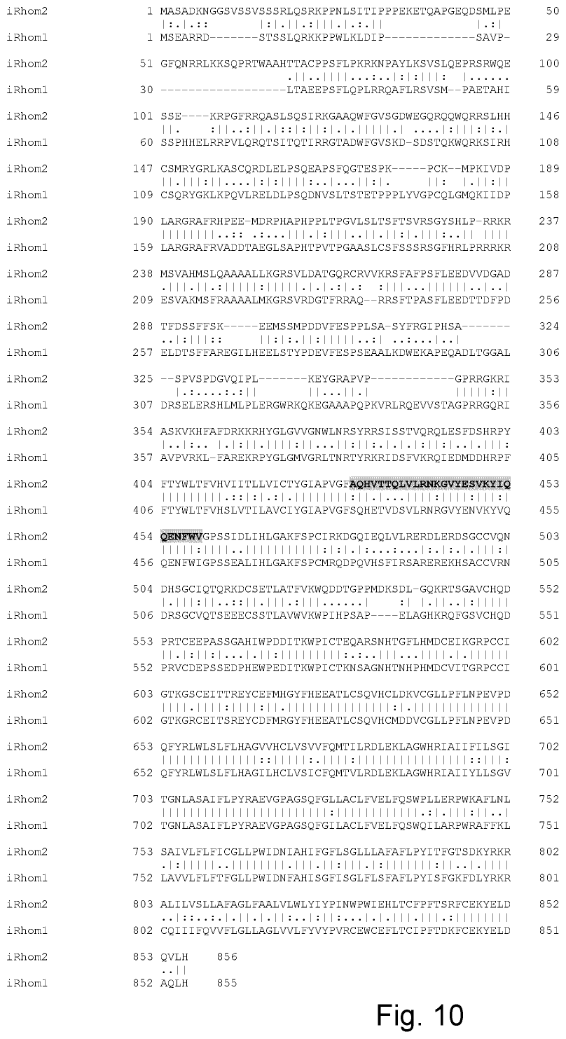

The juxtamembrane domain adjacent to the transmembrane domain 1 (TMD1) is a region that encompasses a stretch of amino acids C-terminally of the first transmembrane domain (TMD1). See FIGS. 8 and 9 for an illustration.

In one embodiment, the juxtamembrane domain adjacent to the transmembrane domain 1 (TMD1) comprises amino acids 431-459 of an amino acid sequence set forth in SEQ ID NO 16, or of an amino acid sequence that has at least 80% sequence identity with SEQ ID NO 16.

In another embodiment, the juxtamembrane domain adjacent to the transmembrane domain 1 (TMD1) comprises amino acids 431-447 of an amino acid sequence set forth in SEQ ID NO 16, or of an amino acid sequence that has at least 80% sequence identity with SEQ ID NO 16.

According to one or more embodiments of the invention, the protein binder binds to an amino acid sequence of human iRhom2 comprising
a) at least the amino acid sequence set forth in SEQ ID NO 3, or
b) an amino acid sequence that has at least 90% sequence identity with SEQ ID NO 3.

In some embodiments, the amino acid sequence that has ≥91%, preferably ≥92%, more preferably ≥93%, ≥94%, ≥95%, ≥96%, ≥97%, ≥98 or most preferably ≥99% sequence identity with SEQ ID NO 3.

In one embodiment, the antibody binds the entire amino acid sequence as set forth above. In another embodiment, the antibody binds also further amino acid sequences of human iRhom2 outside of SEQ ID NO3, or outside of the amino acid sequence that has at least 90% sequence identity with SEQ ID NO 3.

Depending on where the further amino acids are located, the epitope that the antibody binds is linear or conformational.

According to one or more embodiments of the invention, the protein binder binds to one or more amino acid sequences of human iRhom2 each comprising one or more amino acids within the amino acid sequence set forth in SEQ ID NO 3.

In one embodiment, the antibody binds one discrete subsequence within SEQ ID NO 3, which comprises one or more amino acids.

In one embodiment, the antibody binds to two or more discrete subsequences within SEQ ID NO 3, each of which comprises one or more amino acids

According to one or more embodiments of the invention, the protein binder binds to at least one amino acid residue selected from the group consisting of A431, Q432, H433, V434, T435, T436, Q437, L438, V439, L440, R441, N442, K443, G444, V445, Y446, E447, S448, V449, K450, Y451, 1452, Q453, Q454, E455, N456, F457, W458, V459, wherein the numbering of the amino acid residues is relative to the amino acid sequence set forth in SEQ ID NO 16 (human iRhom2)

In one or more embodiments the protein binder binds to ≥2, ≥3, ≥4, ≥5, ≥6, ≥7, ≥8, ≥9, ≥10, ≥11, ≥12, ≥13, ≥14, ≥15, ≥16, ≥17, ≥18, ≥19, ≥20, ≥21, ≥22, ≥23, ≥24, ≥25, ≥26, ≥27, ≥28, or ≥29 amino acid residues from the above list. The respective amino acid residues can be present in a discrete, consecutive sequence, or in two or more clusters within SEQ ID NO 3.

In another embodiment, the protein binder binds to an amino acid sequence of human iRhom2 comprising at least the amino acid sequence set forth in SEQ ID NO 4, or an amino acid sequence that has at least 90% sequence identity with SEQ ID NO 4. The same fallback positions regarding the sequence identity apply.

In one embodiment, the antibody binds the entire amino acid sequence as set forth above. In another embodiment, the antibody binds also further amino acid sequences of human iRhom2 outside of SEQ ID NO 4, or outside of the amino acid sequence that has at least 90% sequence identity with SEQ ID NO 4.

Depending on where the further amino acids are located, the epitope that the antibody binds is linear or conformational.

According to one or more embodiments of the invention, the protein binder binds to one or more amino acid sequences of human iRhom2 each comprising one or more amino acids within the amino acid sequence set forth in SEQ ID NO 4

In one embodiment, the antibody binds one discrete subsequence within SEQ ID NO 4, which comprises one or more amino acids.

In one embodiment, the antibody binds to two or more discrete subsequences within SEQ ID NO 4, each of which comprises one or more amino acids.

According to one or more embodiments of the invention, the protein binder binds to at least one amino acid residue selected from the group consisting of A426, P427, V428, G429, F430, A431, Q432, H433, V434, T435, T436, Q437, L438, V439, L440, R441, N442, K443, G444, V445, Y446, E447, S448, V449, K450, Y451, 1452, Q453, Q454, E455, N456, F457, W458, V459, wherein the numbering of the amino acid residues is relative to the amino acid sequence set forth in SEQ ID NO 16 (human iRhom2)

In one or more embodiments, the protein binder binds to ≥2, ≥3, ≥4, ≥5, ≥6, ≥7, ≥8, ≥9, ≥10, ≥11, ≥12, 13, ≥14, ≥15, ≥16, ≥17, ≥18, ≥19, ≥20, ≥21, ≥22, ≥23, ≥24, ≥25, ≥26, ≥27, ≥28, ≥29, ≥30, ≥31, ≥32, ≥33 or ≥34 amino acid residues from the above list. The respective amino acid residues can be present in a discrete, consecutive sequence, or in two or more clusters within SEQ ID NO 3.

According to one or more embodiments of the invention, the protein binder is not cross-reactive with human iRhom1, or the juxtamembrane domain adjacent to the transmembrane domain 1 (TMD1) thereof.

According to one or more embodiments of the invention, the protein binder is cross-reactive with murine iRhom2, or the juxtamembrane domain adjacent to the transmembrane domain 1 (TMD1) thereof.

According to one or more embodiments of the invention, the protein binder is an antibody in at least one of the formats selected from the group consisting of: IgG, scFv, Fab, (Fab)2.

According to one or more embodiments of the invention, the protein binder is an antibody having an isotype selected from the group consisting of IgG, IgM.

According to one or more embodiments of the invention, the protein binder is a murine, chimerized, humanized, or human antibody.

According to one embodiment of the invention, the protein binder is the antibody 4H8-E3. In one embodiment, the protein binder is an antibody which comprises the variable domains or the CDRs of 4H8-E3.

According to one embodiment of the invention, the protein binder
a) comprises a set of heavy chain/light chain complementarity determining regions (CDR) comprised in the heavy chain/light variable region sequence pair set forth in SEQ ID NOs 33 and 40
b) comprises a set of heavy chain/light chain complementarity determining regions (CDR) comprising the following sequences
   HC CDR1 (SEQ ID NO 34 or 37)
   HC CDR2 (SEQ ID NO 35 or 38)
   HC CDR3 (SEQ ID NO 36 or 39)
   LC CDR1 (SEQ ID NO 41 or 44)
   LC CDR2 (SEQ ID NO 42 or 45), and
   LC CDR3 (SEQ ID NO 43 or 46)
c) comprises the heavy chain/light chain complementarity determining regions (CDR) of b), with the proviso that at least one of the CDRs has up to 3 amino acid substitutions relative to the respective SEQ ID NO 34-39 or 41-46, and/or
d) comprises the heavy chain/light chain complementarity determining regions (CDR) of b) or c), with the proviso that at least one of the CDRs has a sequence identity of ≥66% to the respective SEQ ID NO 34-39 or 41-46,
   wherein the CDRs are embedded in a suitable protein framework so as to be capable to bind to human iRhom2 with sufficient binding affinity and to inhibit or reduce TACE/ADAM17 activity.

These CDRs are the CDRs sets of the antibody 4H8-E3, determined with different approaches (SEQ ID NOs 34-39 determined with the paratome CDR identification tool (http://ofranservices.biu.ac.il/site/services/paratome), and SEQ ID NOs 41-46 determined with in house methods).

As used herein, the term “CDR” or “complementarity determining region” is intended to mean the non-contiguous antigen combining sites found within the variable region of both heavy and light chain polypeptides. These particular regions have been described by Kabat et al. (1977), Kabat et al. (1991), Chothia et al. (1987) and MacCallum et al., (1996) where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. Note that this numbering may differ from the CDRs that acre actually disclosed in the enclosed sequence listing, because CDR definitions vary from case to case.

TABLE 1
CDR definitions

| | Kabat | Chothia | MacCallum |
|---|---|---|---|
| VH CDR1 | 31-35 | 26-32 | 30-35 |
| VH CDR2 | 50-65 | 53-55 | 47-58 |
| VH CDR3 | 95-102 | 96-101 | 93-101 |
| VL CDR1 | 24-34 | 26-32 | 30-36 |
| VL CDR2 | 50-56 | 50-52 | 46-55 |
| VL CDR3 | 89-97 | 91-96 | 89-96 |

As used herein, the term “framework” when used in reference to an antibody variable region is entered to mean all amino acid residues outside the CDR regions within the variable region of an antibody. Therefore, a variable region framework is between about 100-120 amino acids in length but is intended to reference only those amino acids outside of the CDRs.

As used herein, the term “capable to bind to target X with sufficient binding affinity” has to be understood as meaning that respective binding domain binds the target with a K_{D} of 10′ or smaller. K_{D} is the equilibrium dissociation constant, a ratio of k_{off}/kₒₙ, between the protein binder and its antigen. K_{D} and affinity are inversely related. The K_{D} value relates to the concentration of protein binder (the amount of protein binder needed for a particular experiment) and so the lower the K_{D} value (lower concentration) and thus the higher the affinity of the binding domain. The following table shows typical K_{D} ranges of monoclonal antibodies

TABLE 2
K_{D} and Molar Values

| K_{D} value | Molar range |
|---|---|
| 10⁻⁴ to 10⁻⁶ | Micromolar (μM) |
| 10⁻⁷ to 10⁻⁹ | Nanomolar (nM) |
| 10⁻¹⁰ to 10⁻¹² | Picomolar (pM) |
| 10⁻¹³ to 10⁻¹⁵ | Femtomolar (fM) |

Preferably, the protein binder has up to 2 amino acid substitutions, and more preferably up to 1 amino acid substitutions

Preferably, at least one of the CDRs has a sequence identity of ≥67%; ≥68%; ≥69%; ≥70%; ≥71%; ≥72%; ≥73%; ≥74%; ≥75%; ≥76%; ≥77%; ≥78%; ≥79%; ≥80%; ≥81%; ≥82%; ≥83%; ≥84%; ≥85%; ≥86%; ≥87%; ≥88%; ≥89%; ≥90%; ≥91%; ≥92%; ≥93%; ≥94%; ≥95%; ≥96%; ≥97%; ≥98%; ≥99%, and most preferably ≥100% to the respective SEQ ID NO.

As used herein, the term “% sequence identity”, has to be understood as follows: Two sequences to be compared are aligned to give a maximum correlation between the sequences. This may include inserting “gaps” in either one or both sequences, to enhance the degree of alignment. A % identity may then be determined over the whole length of each of the sequences being compared (so-called global alignment), that is particularly suitable for sequences of the same or similar length, or over shorter, defined lengths (so-called local alignment), that is more suitable for sequences of unequal length. In the above context, an amino acid sequence having a “sequence identity” of at least, for example, 95% to a query amino acid sequence, is intended to mean that the sequence of the subject amino acid sequence is identical to the query sequence except that the subject amino acid sequence may include up to five amino acid alterations per each 100 amino acids of the query amino acid sequence. In other words, to obtain an amino acid sequence having a sequence of at least 95% identity to a query amino acid sequence, up to 5% (5 of 100) of the amino acid residues in the subject sequence may be inserted or substituted with another amino acid or deleted. Methods for comparing the identity and homology of two or more sequences are well known in the art. The percentage to which two sequences are identical can for example be determined by using a mathematical algorithm. A preferred, but not limiting, example of a mathematical algorithm is integrated in the BLAST family of programs, e.g. BLAST or NBLAST program and FASTA. Sequences which are identical to other sequences to a certain extent can be identified by these programmes. Furthermore, programs available in the Wisconsin Sequence Analysis Package, version 9.1 for example the programs BESTFIT and GAP, may be used to determine the % identity between two polypeptide sequences. If herein reference is made to an amino acid sequence sharing a particular extent of sequence identity to a reference sequence, then said difference in sequence is preferably due to conservative amino acid substitutions. Preferably, such sequence retains the activity of the reference sequence, e.g. albeit maybe at a slower rate.

Preferably, at least one of the CDRs has been subject to CDR sequence modification, including
affinity maturation
reduction of immunogenicity

Affinity maturation in the process by which the affinity of a given antibody is increased in vitro. Like the natural counterpart, in vitro affinity maturation is based on the principles of mutation and selection. It has successfully been used to optimize antibodies, antibody fragments or other peptide molecules like antibody mimetics. Random mutations inside the CDRs are introduced using radiation, chemical mutagens or error-prone PCR. In addition, the genetic diversity can be increased by chain shuffling. Two or three rounds of mutation and selection using display methods like phage display usually results in antibody fragments with affinities in the low nanomolar range. For principles see Eylenstein et al. (2016), the content of which is incorporated herein by reference.

Humanized antibodies contain murine-sequence derived CDR regions that have been engrafted, along with any necessary framework back-mutations, into human sequence-derived V regions. Hence, the CDRs themselves can cause immunogenic reactions when the humanized antibody is administered to a patient. Methods of reducing immunogenicity caused by CDRs are disclosed in Harding et al. (2010), the content of which is incorporated herein by reference.

According to one embodiment of the invention, the framework is a human VH/VL framework. VH stands for heavy chain variable domain of an IgG shaped antibody, while VL stands for light chain variable domain (kappa or lambda)

According to one embodiment of the invention, the protein binder comprises
a) the heavy chain/light chain variable domains (VD)
   HC VD (SEQ ID NO 33), and
   LC VD (SEQ ID NO 40)
b) the heavy chain/light chain variable domains (VD) of a), with the proviso that
   the HCVD has a sequence identity of ≥80% to the respective SEQ ID NO 33, and/or
   the LCDVD has a sequence identity of ≥80% to the respective SEQ ID NO 40,
c) the heavy chain/light chain variable domains (VD) of a) or b), with the proviso that at least one of the HCVD or LCVD has up to 10 amino acid substitutions relative to the respective SEQ ID NO 33 and/or 40.
   said protein binder still being capable to bind to human iRhom2 with sufficient binding affinity and to inhibit or reduce TACE/ADAM17 activity.

Preferably, the HCVD and/or LCVD has a sequence identity of ≥81%; ≥82%; ≥83%; ≥84%; ≥85%; ≥86%; ≥87%; ≥88%; ≥89%; ≥90%; ≥91%; ≥92%; ≥93%; ≥94%; ≥95%; ≥96%; ≥97%; ≥98%; ≥99%; or most preferably ≥100% to the respective SEQ ID NO.

A “variable domain” when used in reference to an antibody or a heavy or light chain thereof is intended to mean the portion of an antibody which confers antigen binding onto the molecule and which is not the constant region. The term is intended to include functional fragments thereof which maintain some of all of the binding function of the whole variable region. Variable region binding fragments include, for example, functional fragments such as Fab, F(ab)₂, Fv, single chain Fv (scfv) and the like. Such functional fragments are well known to those skilled in the art. Accordingly, the use of these terms in describing functional fragments of a heteromeric variable region is intended to correspond to the definitions well known to those skilled in the art. Such terms are described in, for example, Huston et al., (1993) or Plückthun and Skerra (1990).

According to one embodiment of the invention, at least one amino acid substitution discussed above is a conservative amino acid substitution.

A “conservative amino acid substitution” has a smaller effect on protein binder function than a non-conservative substitution. Although there are many ways to classify amino acids, they are often sorted into six main groups on the basis of their structure and the general chemical characteristics of their R groups.

In one embodiment, a “conservative amino acid substitution” is one in which the amino acid residue is replaced with an amino acid residue having a similar side chain. For example, families of amino acid residues having similar side chains have been defined in the art. These families include amino acids with
basic side chains (e.g., lysine, arginine, histidine),
acidic side chains (e.g., aspartic acid, glutamic acid),
uncharged polar side chains (e.g., glycine, asparagine, glutamine, serine, threonine, tyrosine, cysteine),
nonpolar side chains (e.g., alanine, valine, leucine, isoleucine, proline, phenylalanine, methionine, tryptophan),
beta-branched side chains (e.g., threonine, valine, isoleucine) and
aromatic side chains (e.g., tyrosine, phenylalanine, tryptophan, histidine).

Other conserved amino acid substitutions can also occur across amino acid side chain families, such as when substituting an asparagine for aspartic acid in order to modify the charge of a peptide. Thus, a predicted nonessential amino acid residue in a HR domain polypeptide, for example, is preferably replaced with another amino acid residue from the same side chain family or homologues across families (e.g. asparagine for aspartic acid, glutamine for glutamic acid). Conservative changes can further include substitution of chemically homologous non-natural amino acids (i.e. a synthetic non-natural hydrophobic amino acid in place of leucine, a synthetic non-natural aromatic amino acid in place of tryptophan).

According to one embodiment of the invention, the protein binder has at least one of
target binding affinity of ≥50% to iRhom2, and measured by SPR, compared to that of the protein binder according to the above description, and/or
≥50% of the inhibiting or reducing effect on TACE/ADAM17 activity of the protein binder according to the above description

As used herein the term “binding affinity” is intended to mean the strength of a binding interaction and therefore includes both the actual binding affinity as well as the apparent binding affinity. The actual binding affinity is a ratio of the association rate over the disassociation rate. Therefore, conferring or optimizing binding affinity includes altering either or both of these components to achieve the desired level of binding affinity. The apparent affinity can include, for example, the avidity of the interaction. For example, a bivalent heteromeric variable region binding fragment can exhibit altered or optimized binding affinity due to its valency.

A suitable method for measuring the affinity of a binding agent is through surface plasmon resonance (SPR). This method is based on the phenomenon which occurs when surface plasmon waves are excited at a metal/liquid interface. Light is directed at, and reflected from, the side of the surface not in contact with sample, and SPR causes a reduction in the reflected light intensity at a specific combination of angle and wavelength. Biomolecular binding events cause changes in the refractive index at the surface layer, which are detected as changes in the SPR signal. The binding event can be either binding association or disassociation between a receptor-ligand pair. The changes in refractive index can be measured essentially instantaneously and therefore allows for determination of the individual components of an affinity constant. More specifically, the method enables accurate measurements of association rates (k ₒₙ) and disassociation rates (k_{off}).

Measurements of k ₒₙ and k_{off} values can be advantageous because they can identify altered variable regions or optimized variable regions that are therapeutically more efficacious. For example, an altered variable region, or heteromeric binding fragment thereof, can be more efficacious because it has, for example, a higher kₒₙ valued compared to variable regions and heteromeric binding fragments that exhibit similar binding affinity. Increased efficacy is conferred because molecules with higher kₒₙ values can specifically bind and inhibit their target at a faster rate. Similarly, a molecule of the invention can be more efficacious because it exhibits a lower k_{off} value compared to molecules having similar binding affinity. Increased efficacy observed with molecules having lower k_{off} rates can be observed because, once bound, the molecules are slower to dissociate from their target. Although described with reference to the altered variable regions and optimized variable regions of the invention including, heteromeric variable region binding fragments thereof, the methods described above for measuring associating and disassociation rates are applicable to essentially any protein binder or fragment thereof for identifying more effective binders for therapeutic or diagnostic purposes.

Methods for measuring the affinity, including association and disassociation rates using surface plasmon resonance are well known in the arts and can be found described in, for example, Jonsson and Malmquist, (1992) and Wu et al. (1998). Moreover, one apparatus well known in the art for measuring binding interactions is a BIAcore 2000 instrument which is commercially available through Pharmacia Biosensor, (Uppsala, Sweden).

Preferably said target binding affinity is ≥51%, ≥52%, ≥53%, ≥54%, ≥55%, ≥56%, ≥57%, ≥58%, ≥59%, ≥60%, ≥61%, ≥62%, ≥63%, ≥64%, ≥65%, ≥66%, ≥67%, ≥68%, ≥69%, ≥70%, ≥71%, ≥72%, ≥73%, ≥74%, ≥75%, ≥76%, ≥77%, ≥78%, ≥79%, ≥80%, ≥81%, ≥82%, ≥83%, ≥84%, ≥85%, ≥86%, ≥87%, ≥88%, ≥89%, ≥90%, ≥91%, ≥92%, ≥93%, ≥94%, ≥95%, ≥96%, ≥97%, ≥98%, and most preferably ≥99% compared to that of the reference binding agent.

As used herein, the quantification of the inhibiting or reducing effect on TACE/ADAM17 activity, compared to a benchmark binding agent, can be carried out, e.g., with a respective TNF shedding assay (see., e.g., FIG. 2 and example 5).

According to another aspect of the invention, a protein binder is provided which competes for binding to human iRhom2 with any of the protein binders set forth above.

As regards the format or structure of such protein binders, the same preferred embodiments as set forth above apply. In one embodiment, said protein binder is a monoclonal antibody, or a target-binding fragment or derivative thereof retaining target binding capacities, or an antibody mimetic.

As used herein, the term “competes for binding” is used in reference to one of the antibodies defined by the sequences as above, meaning that the actual protein binder as an activity which binds to the same target, or target epitope or domain or subdomain, as does said sequence defined protein binder, and is a variant of the latter. The efficiency (e.g., kinetics or thermodynamics) of binding may be the same as or greater than or less than the efficiency of the latter. For example, the equilibrium binding constant for binding to the substrate may be different for the two antibodies.

Such competition for binding can be suitably measured with a competitive binding assay. Such assays are disclosed in Finco et al. 2011, the content of which is incorporated herein by reference, and their meaning for interpretation of a patent claim is disclosed in Deng et al 2018, the content of which is incorporated herein by reference.

According to another aspect of the invention, a protein binder is provided that binds to essentially the same, or the same, epitope on iRhom2 as the protein binder according to the above description.

In order to test for this characteristic, suitable epitope mapping technologies are available, including, inter alia,
X-ray co-crystallography and cryogenic electron microscopy (cryo-EM)
Array-based oligo-peptide scanning
Site-directed mutagenesis mapping
High-throughput shotgun mutagenesis epitope mapping
Hydrogen-deuterium exchange
Cross-linking-coupled mass spectrometry

These methods are, inter alia, disclosed and discussed in Banik et al (2010), and DeLisser (1999), the content of which is herein incorporated by reference.

According to another aspect of the invention, a nucleic acid that encodes for a binding agent according to any one of the aforementioned claims.

A given sequence of the encoded binding agent provided, such nucleic acid can have different sequences due to the degeneracy of the genetic code.

Such nucleic acid can be used for pharmaceutic purposes. In such case, it is an RNA-derived molecule that is administered to a patient, wherein the protein expression machinery of the patient expresses the respective binding agent. The mRNA can for example be delivered in suitable liposomes and comprises either specific sequences or modified uridine nucleosides to avoid immune responses and/or improve folding and translation efficiency, sometimes comprising cap modifications at the 5′—and/or 3′ terminus to target them to specific cell types.

Such nucleic acid can be used for transfecting an expression host to then express the actual binding agent. In such case, the molecule can be a cDNA that is optionally integrated into a suitable vector.

According to another aspect of the invention, the use of the protein binder according to the above description is provided (for the manufacture of a medicament) in the treatment of a human or animal subject
being diagnosed for,
suffering from or
being at risk of developing
   an inflammatory condition, or for the prevention of such condition.

According to another aspect of the invention, a pharmaceutical composition comprising the protein binder according to the above description, and optionally one or more pharmaceutically acceptable excipients, is provided.

According to another aspect of the invention, a combination is provided comprising (i) the protein binder according to the above description or the pharmaceutical composition according to the above description and (ii) one or more therapeutically active compounds.

According to another aspect of the invention, a method for treating or preventing an inflammatory condition is provided, which method comprises administration, to a human or animal subject, of (i) the protein binder according to the above description, (ii) the pharmaceutical composition according to the above description or (iii) the combination according to the above description, in a therapeutically sufficient dose.

According to another aspect of the invention, a therapeutic kit of parts is provided, comprising:
a) the composition according to the above description, the pharmaceutical composition according to the above description, or the combination according to the above description,
b) an apparatus for administering the composition, composition or combination, and
c) instructions for use.

### EXAMPLES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′-≥3′.

### Example 1

### Generation of Peptides for Immunization and Peptide Binding ELISA Analyses

Peptides were either synthesized on a parallel peptide synthesizer (peptides 1-5, 7-9 and 1-3b; MultiPep RSi, Intavis AG, Germany), on a microwave peptide synthesizer (peptide 6; Liberty Blue, CEM, USA) or on a custom made continuous flow peptide synthesizer (peptides 10, 11 and 4b) using Fluorenylmethoxycarbonyl (Fmoc)-based Solid Phase Peptide Synthesis. [Chan, W. C., White, P. D. Solid Phase Peptide Synthesis, A Practical Approach (Oxford University Press Inc., New York, 2000]. The sequences were assembled in a stepwise fashion from C to N-terminus using Fmoc-protected L-amino acids with side chain protection groups. Upon completion of the chain assembly peptides were cleaved off the resin with 95% TFA, 4% triethylsilane and 1% water. The crude product was dissolved in 15% acetonitrile in 0.1% aq TFA and purified by reversed phase HPLC using an Orbit C18, 10 μm, 100 Å column (MZ Analysentechnik, Germany). The resulting purified fractions were analyzed by analytical HPLC using a Kinetex EVO C18, 5 μm, 100 Å column (Phenomenex, USA) and by MALDI TOF mass spectrometry (Ultraflex III, Bruker, USA). The fractions were lyophilized yielding the corresponding TFA salt.

For peptide 10 and 11 the linear peptides as identified by mass spectrometry were oxidized to the corresponding cyclic disulfides by DMSO mediated oxidation. For this purpose. the linear peptides were dissolved in 5% acetic acid at a concentration of 1 mg/ml. The pH was adjusted to 6 with (NH4)2CO3 and DMSO was added to a final concentration of 10-20%. The oxidation was allowed to proceed for 24 hours at room temperature. Afterwards the reaction mixture was diluted with solvent A. The product was purified on a reversed phase C18 column and analyzed as described above. Fractions containing the disulfide cyclized peptides were pooled and lyophilized. [Chan, W. C. and White, P. D., Fmoc Solid Phase Peptide Synthesis, A Practical Approach (Oxford University Press Inc., New York, 2000, Chapter 3.3, page 97]

KLH conjugation was performed with pre-activated KLH (Imcejt™ Maleimide Activated mcKLH, Thermo Scientific, USA). Briefly, mcKLH was dissolved with ultrapure water at a concentration of 10 mg/ml. The desired peptide was dissolved at a concentration of 5 mg/mL in Imject™ Maleimide Conjugation Buffer (Thermo Scientific, USA), if necessary 8 M Urea (pH 7.2) was added to dissolve the peptide. The peptide solution was mixed with the mcKLH solution and incubated for 2 to 6 hours at room temperature. The mixture was dialyzed overnight with a 3500-MW cut-off (MWCO) dialysis tube against 400 mL PBS. After dialysis the mixture was diluted with PBS to yield the desired concentration.

Biotinylation was performed with alpha-Biotin-omega-maleimido undeca(ethylene glycol) (Biotin-PEG(11)-mal). The peptides were dissolved in PBS pH 7,4. If necessary, acetonitrile was added to dissolve the peptides. Biotin-PEG(11)-mal was dissolved in DMF and added to the peptide solution in (weight amount=1:1). The reaction was performed overnight and subsequently purified on a reversed phase C18 column and analyzed as described above.

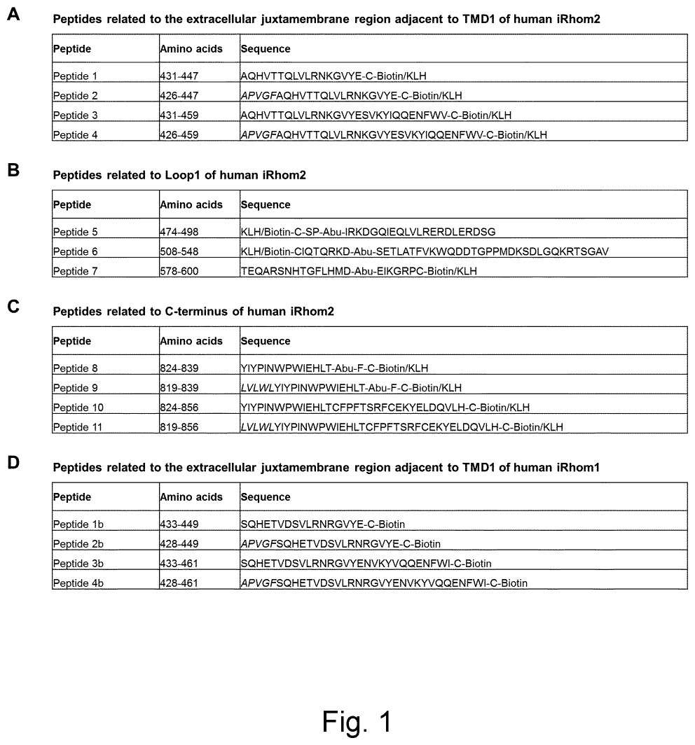

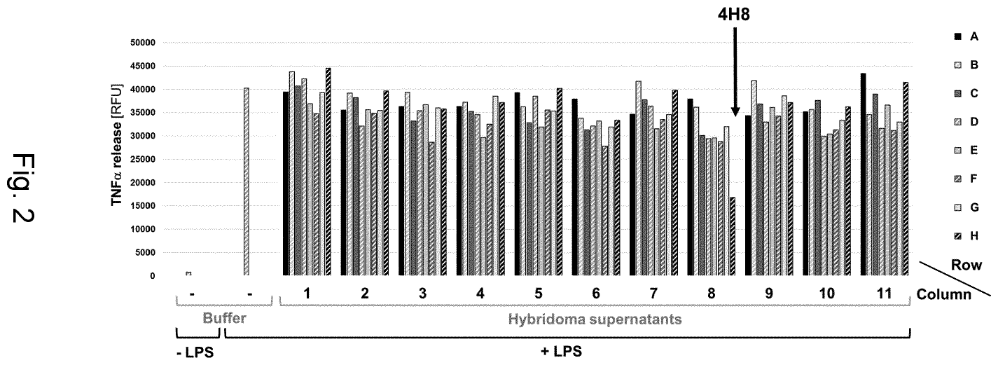

FIG. 1 depicts the peptides used for immunization and/or peptide binding ELISA analyses, indicating their designation, position number and sequence of amino acids with regard to NCBI reference sequences NM_024599.5., NP_078875.4. for human iRhom2 and NCBI reference sequences NM_022450.3., NP_071895.3. for human iRhom1. A terminal cysteine residue added to all peptides except peptides 6 and 7 for coupling to KLH (for immunization) and/or biotin (for peptide binding ELISA analyses) is illustrated by “—C—”. Internal cysteine residues are replaced by alpha-aminobutyric acid (Abu) where indicated. Peptides 1 to 4 correspond to amino acids of TMD1 (highlighted in italics) and the adjacent extracellular juxtamembrane region of human iRhom2 (FIG. 1A). Peptides 5 to 7 resemble sections within the large extracellular loop 1 of human iRhom2 linking TMD1 and TMD2 (FIG. 1B). Peptides 8 to 11 refer to amino acids of TMD7 (highlighted in italics) and the adjacent C-terminal tail of human iRhom2 (FIG. 1C). Peptides 1b to 4b are human iRhom1 homologues of peptides 1 to 4 and, thus, correspond to amino acids of TMD1 (highlighted in italics) and the adjacent extracellular juxtamembrane region of human iRhom1 (FIG. 1D).

### Example 2

### Breeding of iRhom2 Knockout Mice for Immunization

Due to the high sequence homology of human versus mouse iRhom2 protein (referring to the NCBI reference sequence NP_078875.4. for human iRhom2 and the NCBI reference sequence NP_766160.2. for mouse iRhom2, the amino acid sequence identity for the extracellular loops 1, 2, 3 and the C-terminal tail of human versus mouse iRhom2 are calculated as 89.96%, 100.00%, 100.00% and 96.97%, respectively), iRhom2 knockout rather than wild type mice were bred for immunization.

In brief, the Rhbdf2tm1b(KOMP)Wtsi mouse strain (Rhbdf2 is an alternative name for iRhom2) was ordered for resuscitation from the KOMP Mouse Biology Program at University of California, Davis, and resulted in the availability of three heterozygous male mice. These three animals, which were in a C57BL/6N background (C57BL/6N-Rhbdf2tm1b(KOMP)Wtsi), were mated with wild type female mice of a 129Sv/j genetic background to produce heterozygous offspring. These heterozygous mice were mated with one another to generate male and female mice with homozygous knockout of the Rhbdf2 gene. The resulting homozygous Rhbdf2 knockout mouse colony was further expanded for immunization.

### Example 3

### Immunization of Mice and Serum Titer Analysis

Three cohorts of 8 to 10 weeks old male and female iRhom2 knockout mice (as described in Example 2) were immunized with peptide mixes A, B and C, respectively. Mix A consisted of equal amounts of the four keyhole limpet hemocyanin (KLH)-coupled peptides 1, 2, 3 and 4. Mix B was composed of equal amounts of the three KLH-coupled peptides 5, 6 and 7, and Mix C was made up by equal amounts of the four KLH-coupled peptides 8, 9, 10 and 11. Fifty μg of peptide mix were emulsified with 20 μl of GERBU Adjuvant MM™ (GERBU Biotechnik, Germany) and, adjusted with 10 mM HEPES buffer (PH 7,6), were applied for intraperitoneal (IP) administration at a final volume of 100 μl per mouse per injection. Ten mice per cohort were injected every 10 days for five times. Ten days after the fifth injection, blood (serum) was collected and tested for antibody titer .

Assessment of the immune response was conducted by serum antibody titer analysis applying ELISA and FACS methods. With regard to FACS analysis, sera, diluted 1:50 in PBS containing 3% FBS, were tested on murine L929 cells stably expressing human iRhom2 using goat F(ab′)2 anti-Mouse IgG (H+L)-R-phycoerythrin (RPE) conjugate (Dianova, Germany) as secondary antibody. As a negative control. parental L929 cells were used. Tests were performed on an Accuri C6 Plus (BD Biosciences, USA) flow cytometer. Pre-immune serum (“PIS”) taken at day 0 of the immunization protocol served as negative control.

Complementarily, immune sera of all animals were tested in an enzyme-linked immunosorbent assay (ELISA): Sera were diluted 1:500, 1:2,500 and 1:12,500 in PBS containing 1% BSA and tested for binding to plates coated with 1 μg/ml of the respective biotinylated peptide mix through detection with horseradish peroxidase (HRP)-conjugated goat anti-mouse IgG secondary antibody (Southern Biotech, USA). An irrelevant protein (BSA) and the pre-immune sera taken at day 0 of the immunization protocol served as negative controls.

For further boosting of the immune response, the immunization with peptide mixes was extended four days after serum collection by another two injections every 2 weeks and a booster immunization 10 days thereafter. Spleens of selected animals were collected four days after the final boost, lymphocytes were isolated and cryopreserved for subsequent fusions.

### Example 4

### Recovery of Lymphocytes and Fusion for the Generation of Hybridomas

Cryopreserved splenic lymphocytes from 3 selected animals per immunization cohort were thawed and fused group-specifically with Ag8 mouse myeloma cells for the generation of hybridoma cells. Fused cells were plated and grown on 96-well plates in the presence of hypoxanthine-aminopterin-thymidine (HAT) medium. Group-specific fusion allowed retrospective attribution of emerging hybridomas to the respective immunization groups.

### Example 5

### Screen of Hybridoma Supernatants for Candidate Selection

After 14 days of culture, supernatants of hybridoma cells were collected and—instead of being selected for iRhom2 binding antibodies—were subjected to an ELISA-based functional screen for iRhom2 activity-neutralizing antibodies. Since the crucial role of iRhom2 in TACE-mediated release of tumor necrosis factor alpha (TNFα) from macrophages is very well established (McIlwein et al., 2012, Adrain et al., 2012, Siggs et al., 2012), the human TNF-alpha DuoSet ELISA (R&D Systems, USA) was employed to compare the lipopolysaccharide (LPS)-induced release of endogenous TNFα from human THP-1 macrophage cells in the presence and absence of all 5280 peptide immunization-derived hybridoma supernatants.

In brief, on day 1, Nunc black MaxiSorp® 96-well plates (Thermo Fisher Scientific, USA) were coated overnight with 100 μl per well of mouse anti-human TNFα capture antibody (provided as part of the DuoSet ELISA kit) at 4 μg/m1 TBS at 4° C. On day 2, the capture antibody solution was removed and MaxiSorp® plates were blocked overnight with 300 μl per well of TBS, 1% BSA at 4° C. On day 3, 20,000 THP-1 (American Type Culture Collection, USA) cells in 80 μl of normal growth medium were seeded in each well of Greiner CELLSTAR V-bottom 96-well plates (Thermo Fisher Scientific, USA) and pre-incubated with 20 μl of hybridoma supernatants at 37° C., 5% CO₂ for 30 minutes. In case of stimulation controls, 20 μl of standard growth medium instead of hybridoma supernatants were added. Subsequently, cells (except those for unstimulated controls) were stimulated with 20 μl per well of LPS (Sigma-Aldrich, USA) at 300 ng/ml growth medium for a final concentration of 50 ng/ml at 37° C., 5% CO₂ for 2 hours. Afterwards, the 96-well plates were centrifuged to pellet cells. In parallel, blocking buffer was removed from the MaxiSorp® plates and plates were washed 4 times with 350 μl per well of TBS-T (Carl Roth, Germany) on a 96-head plate washer (Tecan Group, Switzerland). To avoid drying-up, 30 μl of TBS were added to each well of the MaxiSorp® plates immediately, followed by the transfer of 70 μl of cell-free supernatant per sample. Additionally, 100 μl of recombinant human TNFα protein (provided as part of the DuoSet ELISA kit) diluted in TB S at defined concentrations were added to the plate as standard references. Thereafter, 100 μl per well of biotinylated goat anti-human TNFα detection antibody (provided as part of the DuoSet ELISA kit) at 50 ng/ml TBS were added and, protected from direct light, plates were incubated at room temperature for 2 hours. After 4 times washing with 350 μl per well of TBS-T (Carl Roth, Germany) on a 96-head plate washer (Tecan Group, Switzerland) and careful removal of all buffer traces after the fourth cycle, 100 μl of streptavidin-AP (R&D Systems, USA) diluted 1:10,000 in TBS were added to each well and, again protected from direct light, plates were incubated at room temperature for 30 minutes. Following another round of 4 times washing with 350 μl per well of TBS-T (Carl Roth, Germany) on a 96-head plate washer (Tecan Group, Switzerland) and careful removal of all buffer traces after the fourth cycle, 100 μl of AttoPhos substrate solution (Promega, USA) was added for incubation in the dark at room temperature for 1 hour. Using an infinite M1000 PRO (Tecan Group, Switzerland) microplate reader, the fluorescence of each well was collected at an excitation wavelength of 435 nm and an emission wavelength of 555 nm.

FIG. 2 shows representative results of these experiments for one 96-well plate demonstrating the effects of peptide immunization-derived hybridoma supernatants on LPS-induced release of TNFα from THP-1 cells. Of the 5280 hybridoma supernatants tested in total, the supernatant collected from the hybridoma cell population of plate number 4, row H, column 8, (4H8) is the only one clearly interfering with LPS-induced TNFα shedding in THP-1 cells.

### Example 6

### Sub-Cloning of the Hybridoma Cell Population 4118

Since the hybridoma cell population 4H8 appeared to be of oligoclonal origin, sub-cloning applying classical liquid dilution technique was performed to isolate monoclonal hybridoma cell pools.

In brief, cells of the hybridoma population 4H8 were counted and the dilution factor to end up with an average of two cells per well of 96-well plates was calculated. Cells were diluted accordingly and wells with growth of a single cell population were identified through microscopy. After expansion of these monoclonal hybridoma populations for approximately 3 weeks, supernatants were collected and compared for inhibitory effects on LPS-induced release of TNFα from THP-1 cells as described in Example 5. Three 4H8 sub-clones, designated 4H8-D4, 4H8-E3 and 4H8-G8, turned out to significantly interfere with TNFα shedding and, thus, were expanded and stocked.

### Example 7

### Purification of Antibody from the Hybridoma Sub-Clone 4118-E3

In this example, the purification of antibody from supernatant of the hybridoma sub-clone 4H8-E3 applying affinity chromatography is described.

In brief, although protein G sepharose is primarily recommended for immobilization of IgG antibodies and described to be less suitable for binding of IgM antibodies, protein G sepharose columns were empirically found to result in good yields of both antibody isotypes. Thus, supernatants collected from the hybridoma sub-clone 4H8-E3 were pooled and loaded on an equilibrated protein G sepharose prepacked gravity-flow column (Protein G GraviTrap™, GE Healthcare, UK) for antibody capturing. Afterwards, columns were washed once with binding buffer and trapped antibody was eluted with elution buffer (both buffers are provided as part of the Ab Buffer Kit; GE Healthcare, UK). Next, the eluate fraction was desalted using PD Miditrap G-25 columns (GE Healthcare, UK), and purified samples were concentrated via Amicon Ultra-4 Centrifugal Filter Units with a cutoff at 30 kDa (Sigma-Aldrich, USA). Finally, the concentration of purified protein was determined applying a NanoDrop 2000/c spectrophotometer (Thermo Fisher Scientific, USA).

Example 8: Isotype determination of the antibody 4118-E3 of the invention

As a next step, a mouse IgG/IgM ELISA was performed to determine the isotype of the purified antibody 4H8-E3 of the invention. In brief, on day 1, Nunc black MaxiSorp® 96-well plates (Thermo Fisher Scientific, USA) were coated overnight with 100 μl per well of goat anti mouse IgG+IgM (H+L) capture antibody (Sigma-Aldrich, USA) at 1 μg/ml TBS at 4° C. On day 2, the capture antibody solution was removed and MaxiSorp® plates were blocked with 300 μl per well of Pierce protein-free (TBS) blocking buffer (Thermo Fisher Scientific, USA) at room temperature for 1 hour. The blocking buffer was then removed and plates were washed 3 times with 350 μl per well of TBS-T (Carl Roth, Germany) on a 96-head plate washer (Tecan Group, Switzerland). Afterwards, 100 μl per well of TBS as blank and negative control, mouse IgG (Thermo Fisher Scientific, USA) and mouse IgM (Sigma-Aldrich, USA) antibody at defined concentrations (both 1:2 titrations starting at 1 μg/ml TBS) as standard references, mouse IgG (Thermo Fisher Scientific, USA) and mouse IgM (Sigma-Aldrich, USA) antibody at 3 μg/ml TBS each as positive and specificity controls, and the purified antibody 4H8-E3 of the invention at 3 μg/ml TBS were added to wells and incubated at room temperature for 2 hours. Subsequently, the plates again were washed 3 times with 350 μl per well of TBS-T (Carl Roth, Germany) on a 96-head plate washer (Tecan Group, Switzerland). For isotype detection, one half of the sample each were, protected from direct light, incubated with 100 μl per well of AP-conjugated goat anti mouse IgM (Sigma-Aldrich, USA) or AP-conjugated goat anti mouse IgG F(ab′)2 Fragment (Dianova, Germany) detection antibodies diluted 1:5,000 in TBS for 1.5 hours at room temperature. Following another round of 3 washing steps with 350 μl per well of TBS-T (Carl Roth, Germany) on a 96-head plate washer (Tecan Group, Switzerland) and careful removal of all buffer traces after the third cycle, 100 μl of AttoPhos substrate solution (Promega, USA) were added for incubation in the dark and at room temperature for 10 minutes. Using an infinite M1000 PRO (Tecan Group, Switzerland) microplate reader, the fluorescence of each well was collected at an excitation wavelength of 435 nm and an emission wavelength of 555 nm.

FIG. 3 shows representative results of this experiment clearly demonstrating the antibody 4H8-E3 of the invention to be of mouse IgM isotype.

### Example 9

### Determination of the Target Region Recognized by the Antibody 4118-E3 of the Invention

Next, peptide binding ELISA analyses were performed to verify whether the purified antibody 4H8-E3 of the invention recognizes any of the peptides that were administered to those animals the hybridoma clone 4H8 was derived from, thereby shedding light on the target region being recognized by the antibody 4H8-E3 of the invention.

In brief, on day 1, Nunc black MaxiSorp® 96-well plates (Thermo Fisher Scientific, USA) were coated overnight with 100 μl per well of single biotinylated peptides 1 to 11 as well as mixes of peptides 1 to 4 (Mix A), 5 to 7 (Mix B), and 8 to 11 (Mix C) at 10 μg/ml TBS each (thus, the final concentration of each peptide in mixes 1 to 4 and 8 to 11 was 2.5 μg/ml versus 3.3 μg/ml in mix 5 to 7) at 4° C. On day 2, peptide solutions were removed and MaxiSorp® plates were blocked with 300 μl per well of Pierce protein-free (TBS) blocking buffer (Thermo Fisher Scientific, USA) at room temperature for 1.5 hours. The blocking buffer was then removed and plates were washed 4 times with 350 μl per well of TBS-T (Carl Roth, Germany) on a 96-head plate washer (Tecan Group, Switzerland). Afterwards, 100 μl per well of TBS as blank control, mouse anti-biotin antibody (clone BN-34, Sigma-Aldrich, USA) at 0.3 μg/ml TBS as coating control, the purified antibody 4H8-E3 of the invention at 3 μg/ml TBS, and mouse IgM antibody (clone MOPC 104E, Sigma-Aldrich, USA) as isotype control to the purified antibody 4H8-E3 of the invention at 3 μg/ml TBS were added to wells pre-coated with single peptides 1 to 11 or respective mixes and incubated at room temperature for 4 hours. Subsequently, the plates were washed 4 times with 350 μl per well of TBS-T (Carl Roth, Germany) on a 96-head plate washer (Tecan Group, Switzerland) again and, protected from direct light, were incubated with 100 μl per well of AP-conjugated goat anti mouse IgG/IgG/IgM F(ab′)2 fragment (Sigma-Aldrich, USA) diluted 1:2,000 in TBS for 1 hour at room temperature. Following another round of 4 washing steps with 350 μl per well of TBS-T (Carl Roth, Germany) on a 96-head plate washer (Tecan Group, Switzerland) and careful removal of all buffer traces after the fourth cycle, 100 μl of AttoPhos substrate solution (Promega, USA) were added for incubation in the dark and at room temperature for 1 hour. Using an infinite M1000 PRO (Tecan Group, Switzerland) microplate reader, the fluorescence of each well was collected at an excitation wavelength of 435 nm and an emission wavelength of 555 nm.

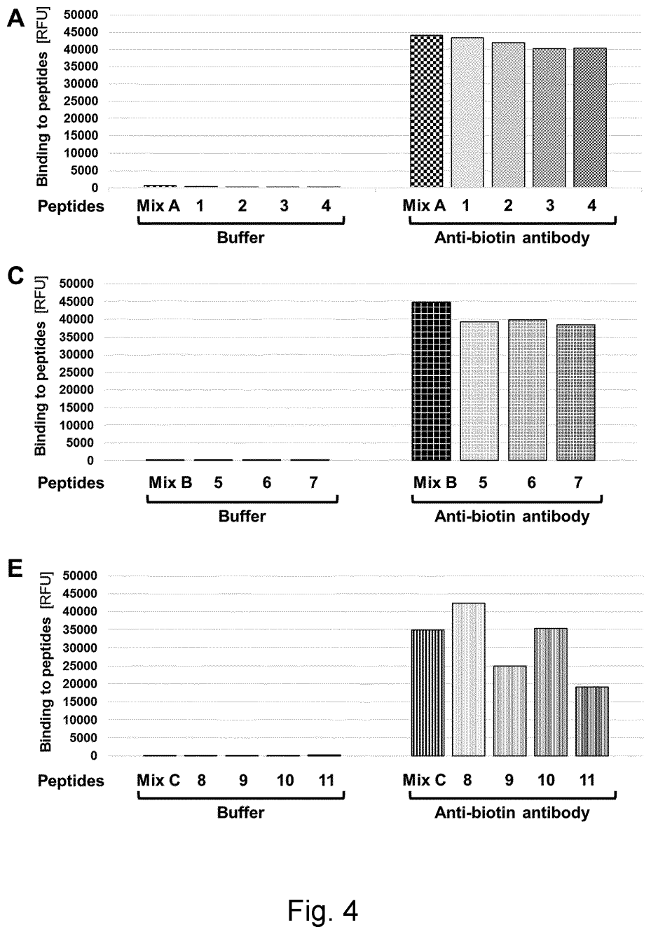

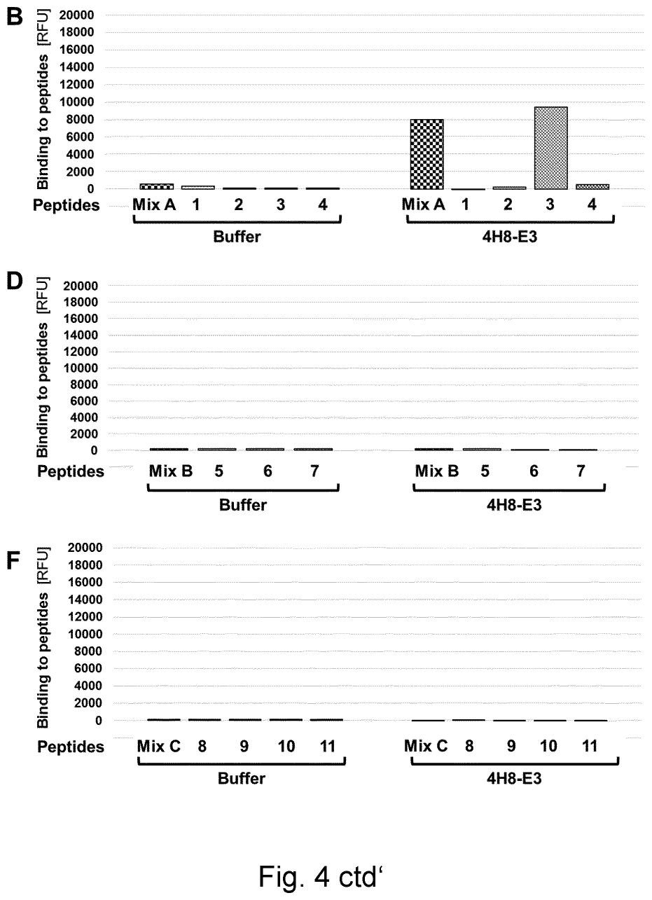

FIG. 4 shows representative results of this experiment. Coating controls confirm the abundance of biotinylated peptides immobilized individually or as peptide mixes (FIG. 4A, C, E). In line with the clone 4H8 to originate from mice immunized with the mix of peptides 1 to 4 (Mix A), the antibody 4H8-E3 of the invention shows no binding to peptides 5, 6 and 7 resembling different sections of the large extracellular loop (FIG. 4D) or peptides 8, 9, 10, and 11 reflecting the C-terminal tail of human iRhom2 (FIG. 4F), regardless whether these peptides were coated individually or as mixes. In contrast, strong binding of the antibody 4H8-E3 of the invention to Mix A consisting of peptides 1, 2, 3, and 4 as well as to the single peptide 3 was demonstrated (FIG. 4B) revealing the epitope recognized by the antibody 4H8-E3 of the invention to be localized within amino acids 431 to 459 of the extracellular juxtamembrane domain of human iRhom2. Data on the antibody 4H8-E3 of the invention are shown after normalization to the IgM isotype control.

### Example 10

### Assessment of Binding Specificity of the Antibody 4118-E3 of the Invention

Another series of peptide binding ELISA experiments was conducted to address the specificity of the purified antibody 4H8-E3 of the invention, i.e. to question whether this antibody specifically recognizes peptides, in particular peptide 3, resembling the extracellular juxtamembrane region adjacent to the TMD1 of human iRhom2, or whether the antibody 4H8-E3 of the invention also binds to peptides reflecting the homologous region of the closely related family member human iRhom1.

In brief, on day 1, Nunc black MaxiSorp® 96-well plates (Thermo Fisher Scientific, USA) were coated overnight with 100 μl per well of single biotinylated peptides 1 to 4, Mix A consisting of peptides 1 to 4, single biotinylated peptides 1b to 4b, and Mix D consisting of peptides 1b to 4b at 10 μg/m1 PBS each (thus, the final concentration of each peptide in both mixes was 2.5 μg/m1) at 4° C. On day 2, peptide solutions were removed and MaxiSorp® plates were blocked with 300 μl per well of Pierce protein-free (TBS) blocking buffer (Thermo Fisher Scientific, USA) at room temperature for 1.5 hours. The blocking buffer was then removed and plates were washed 4 times with 350 μl per well of PBS-T (Carl Roth, Germany) on a 96-head plate washer (Tecan Group, Switzerland). Afterwards, 100 μl per well of PBS as blank control, mouse anti-biotin antibody (clone BN-34, Sigma) at 0.3 μg/ml PBS as coating control, the purified antibody 4H8-E3 of the invention at 3 μg/ml PBS, and mouse IgM antibody (clone PFR-03, Sigma) as isotype control to the purified antibody 4H8-E3 of the invention at 3 μg/ml PBS were added to wells pre-coated with single peptides 1 to 4, 1b to 4b or respective mixes and incubated at room temperature for 4 hours. Subsequently, the plates were washed 4 times with 350 μl per well of PBS-T (Carl Roth, Germany) on a 96-head plate washer (Tecan Group, Switzerland) again and, protected from direct light, were incubated with 100 μl per well of AP-conjugated goat anti mouse IgG/IgG/IgM F(ab′)2 fragment (Sigma-Aldrich, USA) diluted 1:2,000 in PBS for 1 hour at room temperature. Following another round of 4 washing steps with 350 μl per well of PBS-T (Carl Roth, Germany) on a 96-head plate washer (Tecan Group, Switzerland) and careful removal of all buffer traces after the fourth cycle, 100 μl of AttoPhos substrate solution (Promega, USA) were added for incubation in the dark and at room temperature for 1 hour. Using an infinite M1000 PRO (Tecan Group, Switzerland) microplate reader, the fluorescence of each well was collected at an excitation wavelength of 435 nm and an emission wavelength of 555 nm.

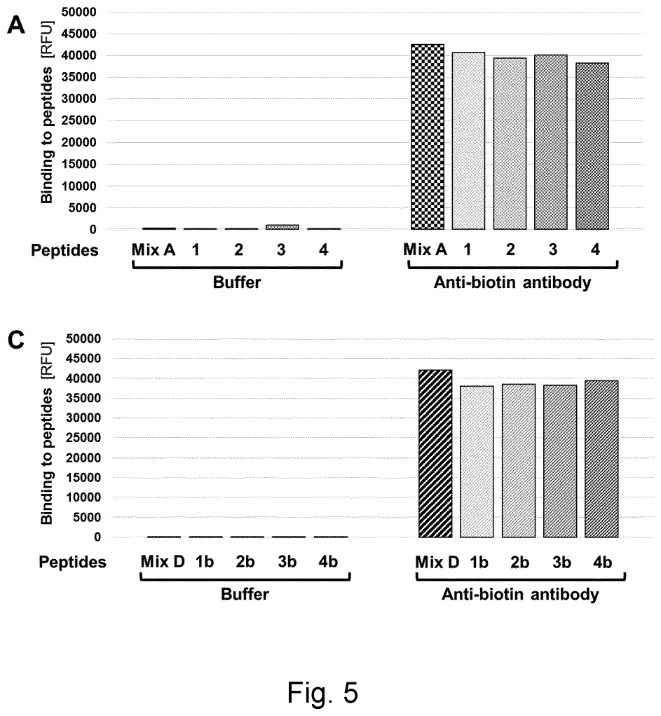

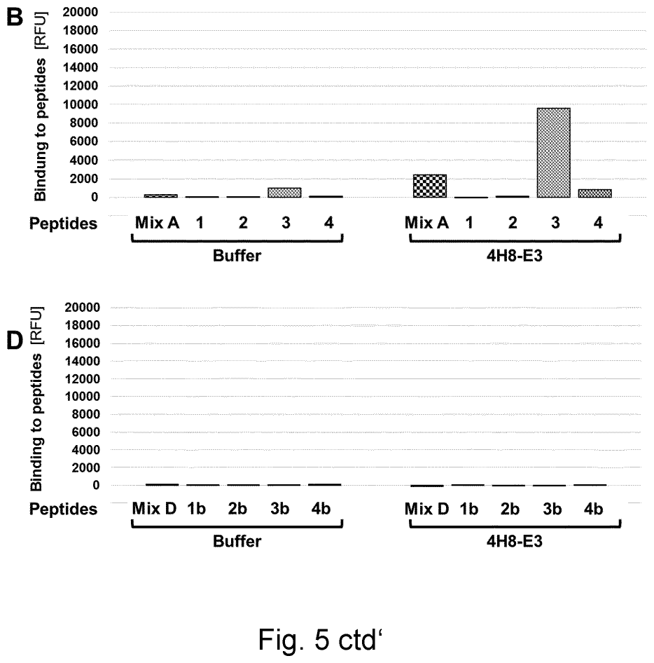

FIG. 5 shows representative results of this experiment. Coating controls again confirm the abundance of biotinylated peptides immobilized individually or as peptide mixes (FIG. 5A, C). Binding of the antibody 4H8-E3 of the invention to Mix A consisting of peptides 1, 2, 3, and 4 and, in particular, the single peptide 3 resembling amino acids 431 to 459 of the extracellular juxtamembrane domain of human iRhom2 was confirmed (FIG. 5B). In contrast, the antibody 4H8-E3 of the invention does not bind at all to Mix D consisting of or individually coated peptides 1b, 2b, 3b and 4b reflecting the homologous amino acid sequences within the related family member human iRhom1 (FIG. 5D) providing evidence for the antibody 4H8-E3 of the invention to specifically bind to human iRhom2 and, thus, not to recognize the homologous section in human iRhom1. Data on the antibody 4H8-E3 of the invention are shown after normalization to the IgM isotype control.

### Example 11

### Analysis of Inhibitory Effects of the Antibody 4118-E3 of the Invention on LPS-Induced TNFα Shedding In Vitro.

In the following study, ELISA-based TNFα release assays were performed to verify the inhibitory effects of the purified antibody 4H8-E3 of the invention on LPS-induced release of endogenous TNFα from human THP-1 macrophage cells.

In brief, on day 1, Nunc black MaxiSorp® 96-well plates (Thermo Fisher Scientific, USA) were coated overnight with 100 μl per well of mouse anti-human TNFα capture antibody (provided as part of the DuoSet ELISA kit) at 4 μg/ml TBS at 4° C. On day 2, the capture antibody solution was removed and MaxiSorp® plates were blocked with 300 μl per well of TBS, 1% BSA at room temperature for 3 hours. Meanwhile, 20,000 THP-1 (American Type Culture Collection, USA) cells in 80 μl of normal growth medium were seeded in each well of Greiner CELLSTAR V-bottom 96-well plates (Thermo Fisher Scientific, USA) and pre-incubated with 20 μl per well of standard growth medium supplemented with Batimastat (BB94, Abcam, UK) at 50 μM as positive control (for a final concentration of 10 μM in the resulting 100 μl sample volume), mouse IgM antibody (clone PFR-03, Sigma-Aldrich, USA) at 50 μg/ml as isotype control (for a final concentration of 10 μg/m1 in the resulting 100 μl sample volume) or purified antibody 4H8-E3 of the invention at 50 μg/ml (for a final concentration of 10 μg/ml in the resulting 100 μl sample volume) at 37° C., 5% CO2 for 30 minutes. In case of stimulation controls, 20 μl of standard growth medium without test articles were added. Subsequently, cells (except those for unstimulated controls) were stimulated with 20 μl per well of LPS (Sigma-Aldrich, USA) at 300 ng/ml growth medium for a final concentration of 50 ng/ml at 37° C., 5% CO₂ for 2 hours. Afterwards, the 96-well plates were centrifuged to pellet cells. In parallel, blocking buffer was removed from the MaxiSorp® plates and plates were washed 4 times with 350 μl per well of TBS-T (Carl Roth, Germany) on a 96-head plate washer (Tecan Group, Switzerland). To avoid drying-up, 30 μl of TBS were added to each well of the MaxiSorp® plates immediately, followed by the transfer of 70 μl of cell-free supernatant per sample. Additionally, 100 μl of recombinant human TNFα protein (provided as part of the DuoSet ELISA kit) diluted in TBS at defined concentrations were added to the plate as standard references. Thereafter, 100 μl per well of biotinylated goat anti-human TNFα detection antibody (provided as part of the DuoSet ELISA kit) at 50 ng/ml TBS were added and, protected from direct light, plates were incubated at room temperature for 2 hours. After 4 times washing with 350 μl per well of TBS-T (Carl Roth, Germany) on a 96-head plate washer (Tecan Group, Switzerland) and careful removal of all buffer traces after the fourth cycle, 100 μl of streptavidin-AP (R&D Systems, USA) diluted 1:10,000 in TBS were added to each well and, again protected from direct light, plates were incubated at room temperature for 30 minutes. Following another round of 4 times washing with 350 μl per well of TBS-T (Carl Roth, Germany) on a 96-head plate washer (Tecan Group, Switzerland) and careful removal of all buffer traces after the fourth cycle, 100 μl of AttoPhos substrate solution (Promega, USA) was added for incubation in the dark at room temperature for 1 hour. Using an infinite M1000 PRO (Tecan Group, Switzerland) microplate reader, the fluorescence of each well was collected at an excitation wavelength of 435 nm and an emission wavelength of 555 nm.

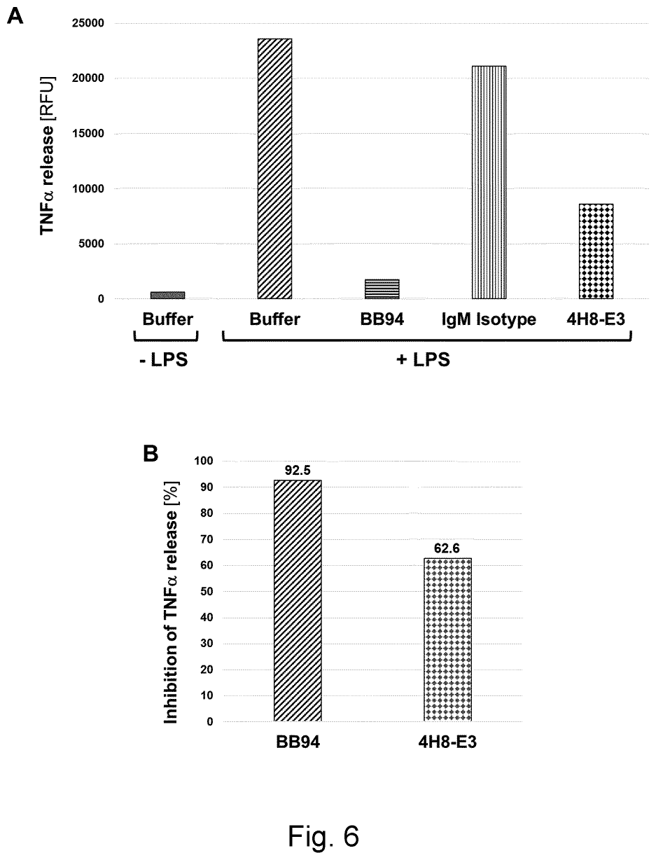

FIG. 6 shows representative results of this experiment demonstrating the effects of test articles on LPS-induced release of TNFα from THP-1 cells in absolute numbers (FIG. 6A) and percent inhibition (FIG. 6B). While Batimastat (BB94) as a small molecule inhibitor of metalloproteinases serves as positive control and results in 92.5% inhibition of LPS-induces release of TNFα, the presence of IgM isotype control has no significant effect on TNFα shedding. In contrast, the equal concentration of the purified antibody 4H8-E3 of the invention inhibits LPS-induced release of TNFα from THP-1 cells by 62.6%.

### Example 12

### Determination of the IC50 of the Antibody 4118-E3 of the Invention on LPS-Induced TNFα Shedding In Vitro.

Expanding the functional analyses, ELISA-based TNFα release assays were performed to determine the half maximal inhibitory concentration (IC50) for the purified antibody 4H8-E3 of the invention on LPS-induced release of endogenous TNFα from human THP-1 macrophage cells.

In brief, on day 1, Nunc black MaxiSorp® 96-well plates (Thermo Fisher Scientific, USA) were coated overnight with 100 μl per well of mouse anti-human TNFα capture antibody (provided as part of the DuoSet ELISA kit) at 4 μg/ml TBS at 4° C. On day 2, the capture antibody solution was removed and MaxiSorp® plates were blocked with 300 μl per well of TBS, 1% BSA at room temperature for 3 hours. Meanwhile, 20,000 THP-1 (American Type Culture Collection, USA) cells in 80 μl of normal growth medium were seeded in each well of Greiner CELLSTAR V-bottom 96-well plates (Thermo Fisher Scientific, USA) and pre-incubated with 20 μl per well of standard growth medium supplemented with the purified antibody 4H8-E3 of the invention at approximately 400.00 μg/ml, 307.69 μg/ml, 236.68 μg/ml, 182.06 μg/ml, 140.05 μg/ml, 107.73 μg/ml, 82.87 μg/ml, 63.74 μg/ml, 49.03 μg/ml, 37.71 μg/ml, 29.01 μg/ml, 22.31 μg/ml, 17.16 μg/ml, 13.20 μg/ml, 10.15 μg/ml, 7.81 μg/ml, 6.01 μg/ml, 4.62 μg/ml, 3.55 μg/ml, 2.73 μg/ml, 2.10 μg/ml, 1.61 μg/ml, 1.24 μg/ml, 0.95 μg/ml, 0.73 μg/ml, 0.56 μg/ml, and 0.43 μg/ml (for a final concentration of approximately 80.00 μg/ml, 61.53 μg/ml, 47.33 μg/ml, 36.41 μg/ml, 28.01 μg/ml, 21.54 μg/ml, 16.57 μg/ml, 12.74 μg/ml, 9.80 μg/ml, 7.54 μg/ml, 5.80 μg/ml, 4.46 μg/ml, 3.43 μg/ml, 2.64 μg/ml, 2.03 μg/ml, 1.56 μg/ml, 1.20 μg/ml, 0.92 μg/ml, 0.71 μg/ml, 0.54 μg/ml, 0.42 μg/ml, 0.32 μg/ml, 0.24 μg/ml, 0.19 μg/ml, 0.14 μg/ml, 0.11 μg/ml, and 0.08 μg/ml, respectively, in the resulting 100 μl sample volume) at 37° C., 5% CO₂ for 30 minutes. Subsequently, cells (except those for unstimulated controls) were stimulated with 20 μl per well of LPS (Sigma-Aldrich, USA) at 300 ng/ml growth medium for a final concentration of 50 ng/ml at 37° C., 5% CO₂ for 3 hours. Afterwards, the 96-well plates were centrifuged to pellet cells. In parallel, blocking buffer was removed from the MaxiSorp® plates and plates were washed 4 times with 350 μl per well of TBS-T (Carl Roth, Germany) on a 96-head plate washer (Tecan Group, Switzerland). To avoid drying-up, 30 μl of TBS were added to each well of the MaxiSorp® plates immediately, followed by the transfer of 70 μl of cell-free supernatant per sample. Additionally, 100 μl of recombinant human TNFα protein (provided as part of the DuoSet ELISA kit) diluted in TBS at defined concentrations were added to the plate as standard references. Thereafter, 100 μl per well of biotinylated goat anti-human TNFα detection antibody (provided as part of the DuoSet ELISA kit) at 50 ng/ml TBS were added and, protected from direct light, plates were incubated at room temperature for 2 hours. After 4 times washing with 350 μl per well of TBS-T (Carl Roth, Germany) on a 96-head plate washer (Tecan Group, Switzerland) and careful removal of all buffer traces after the fourth cycle, 100 μl of streptavidin-AP (R&D Systems, USA) diluted 1:10,000 in TBS were added to each well and, again protected from direct light, plates were incubated at room temperature for 30 minutes. Following another round of 4 times washing with 350 μl per well of TBS-T (Carl Roth, Germany) on a 96-head plate washer (Tecan Group, Switzerland) and careful removal of all buffer traces after the fourth cycle, 100 μl of AttoPhos substrate solution (Promega, USA) was added for incubation in the dark at room temperature for 1 hour. Using an infinite M1000 PRO (Tecan Group, Switzerland) microplate reader, the fluorescence of each well was collected at an excitation wavelength of 435 nm and an emission wavelength of 555 nm.

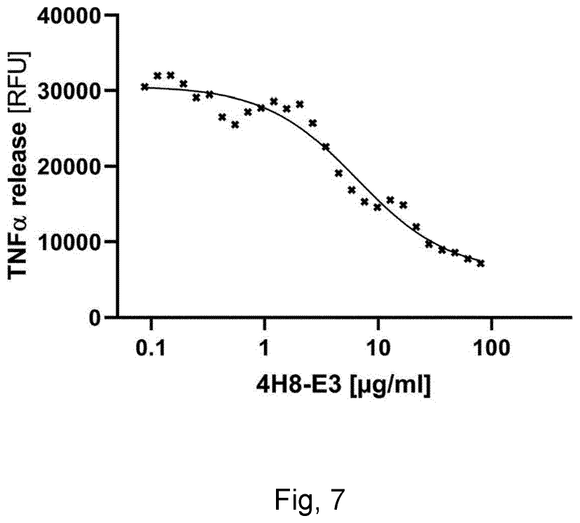

FIG. 7 shows representative results of this experiment. Titration of the purified antibody 4H8-E3 of the invention leads to a concentration-dependent inhibition of TNFα release from THP-1 cells. Applying Prism8 software (GraphPad Software, USA), the respective IC50 value for the antibody 4H8-E3 of the invention is calculated as 6.48 nM.

### REFERENCES

Köhler, G. & Milstein, C. (1975): Continuous cultures of fused cells secreting antibody of predefined specificity. In: Nature. Bd. 256, S. 495-497. Jonsson and Malmquist, Advances in Biosnsors, 2:291-336 (1992)
Wu et al. Proc. Natl. Acad. Sci. USA, 95:6037-6042 (1998)
Banik, SSR; Doranz, B J (2010). “Mapping complex antibody epitopes”. Genetic Engineering & Biotechnology News. 3 (2): 25-8
DeLisser, H M (1999). Epitope mapping. Methods Mol Biol. 96. pp. 11-20
Finco et al, Comparison of competitive ligand-binding assay and bioassay formats for the measurement of neutralizing antibodies to protein therapeutics. J Pharm Biomed Anal. 2011 Jan 25;54(2):351-8. doi: 10.1016/j.jpba.2010.08.029. Epub 2010 Sep 21
Deng et al., Enhancing antibody patent protection using epitope mapping information MAbs. 2018 Feb-Mar; 10(2): 204-209
Huston et al., Cell Biophysics, 22:189-224 (1993);
Plückthun and Skerra, Meth. Enzymol., 178:497-515 (1989) and in Day, E. D., Advanced Immunochemistry, Second Ed., Wiley-Liss, Inc., New York, N.Y. (1990)
Harding, The immunogenicity of humanized and fully human antibodies. MAbs. 2010 May-Jun; 2(3): 256-265.
Eylenstein, et al, Molecular basis of in vitro affinity maturation and functional evolution of a neutralizing anti-human GM-CSF antibody, mAbs, 8:1, 176-186 (2016)
Kabat et al., J. Biol. Chem. 252, 6609-6616 (1977) and Kabat et al., Sequences of protein of immunological interest. (1991)
Chothia et al., J. Mol. Biol. 196:901-917 (1987)
MacCallum et al., J. Mol. Biol. 262:732-745 (1996)

### SEQUENCES

The following sequences form part of the disclosure of the present application. A WIPO ST 25 compatible electronic sequence listing is provided with this application, too. For the avoidance of doubt, if discrepancies exist between the sequences in the following table and the electronic sequence listing, the sequences in this table shall be deemed to be the correct ones.

| SEQ | | |
|---|---|---|
| ID | Sequence | Comment |
| 1 | AQHVTTQLVLRNKGVYEC | Human iRhom2 Juxtamembrane |
| | | domain of TMD1; AA431-447 |
| | | (″immunization peptide 1″) |
| 2 | APVGFAQHVTTQLVLRNKGVYEC | Human iRhom2 Juxtamembrane |
| | | domain of TMD1; AA426-447 |
| | | (″immunization peptide 2″) |
| 3 | AQHVTTQLVLRNKGVYESVKYIQQENFWVC | Human iRhom2 Juxtamembrane |
| | | domain of TMD1; AA431-459 |
| | | (″immunization peptide 3″) |
| 4 | APVGFAQHVTTQLVLRNKGVYESVKYIQQENFWVC | Human iRhom2 Juxtamembrane |
| | | domain of TMD1; AA426-459 |
| | | (″immunization peptide 4″) |
| 5 | CSPXIRKDGQIEQLVLRERDLERDSG | Human iRhom2 Loop 1; AA474-498 |
| | | (″immunization peptide 5″); X = |
| | | aminobutyrate |
| 6 | CIQTQRKDXSETLATFVKWQDDTGPPMDKsDLGQKRTSGAV | Human iRhom2 Loop 1; AA508-548 |
| | | (″immunization peptide 6″); X = |
| | | aminobutyrate |
| 7 | TEQARSNHTGFLHMDXEIKGRPC | Human iRhom2 Loop 1; AA578-600 |
| | | (″immunization peptide 7″); X = |
| | | aminobutyrate |
| 8 | YIYPINWPWIEHLTXFC | Human iRhom2 C-Terminus; AA824- |
| | | 839 (″immunization peptide 8″); X = |
| | | aminobutyrate |
| 9 | LVLWLYIYPINWPWIEHLTXFC | Human iRhom2 C-Terminus; AA819- |
| | | 839 (″immunization peptide 9″); X = |
| | | aminobutyrate |
| 10 | YIYPINWPWIEHLTCFPFTSRFCEKYELDQVLHC | Human iRhom2 C-Terminus; AA824- |
| | | 856 (″immunization peptide 10″) |
| 11 | LVLWLYIYPINWPWIEHLTCFPFTSRFCEKYELDQVLHC | Human iRhom2 C-Terminus; AA819- |
| | | 856 (″immunization peptide 11″) |
| 12 | SQHETVDSVLRNRGVYEC | Human iRhom1 Juxtamembrane |
| | | domain of TMD1; AA433- |
| | | 449(″peptide 1b″) |
| 13 | APVGFSQHETVDSVLRNRGVYEC | Human iRhom1 Juxtamembrane |
| | | domain of TMD1; AA428- |
| | | 449(″peptide 2b″) |
| 14 | SQHETVDSVLRNRGVYENVKYVQQENFWIC | Human iRhom1 Juxtamembrane |
| | | domain of TMD1; AA433- |
| | | 461(″peptide 3b″) |
| 15 | APVGFSQHETVDSVLRNRGVYENVKYVQQENFWIC | Human iRhom1 Juxtamembrane |
| | | domain of TMD1; AA428- |
| | | 461(″peptide 4b″) |
| 16 | MASADKNGGSVSSVSSSRLQSRKPPNLSITIPPPEKETQAP | >NP_078875.4 human iRhom2 |
| | GEQDSMLPEGFQNRRLKKSQPRTWAAHTTACPPSFLPKRKN | isoform 1 |
| | PAYLKSVSLQEPRSRWQESSEKRPGFRRQASLSQSIRKGAA | |
| | QWFGVSGDWEGQRQQWQRRSLHHCSMRYGRLKASCQRDLEL | |
| | PSQEAPSFQGTESPKPCKMPKIVDPLARGRAFRHPEEMDRP | |
| | HAPHPPLTPGVLSLTSFTSVRSGYSHLPRRKRMSVAHMSLQ | |
| | AAAALLKGRSVLDATGQRCRVVKRSFAFPSFLEEDVVDGAD | |
| | TFDSSFFSKEEMSSMPDDVFESPPLSASYFRGIPHSASPVS | |
| | PDGVQIPLKEYGRAPVPGPRRGKRIASKVKHFAFDRKKRHY | |
| | GLGVVGNWLNRSYRRSISSTVQRQLESFDSHRPYFTYWLTF | |
| | VHVIITLLVICTYGIAPVGFAQHVTTQLVLRNKGVYESVKY | |
| | IQQENFWVGPSSIDLIHLGAKFSPCIRKDGQIEQLVLRERD | |
| | LERDSGCCVQNDHSGCIQTQRKDCSETLATFVKWQDDTGPP | |
| | MDKSDLGQKRTSGAVCHQDPRTCEEPASSGAHIWPDDITKW | |
| | PICTEQARSNHTGFLHMDCEIKGRPCCIGTKGSCEITTREY | |
| | CEFMHGYFHEEATLCSQVHCLDKVCGLLPFLNPEVPDQFYR | |
| | LWLSLFLHAGVVHCLVSVVFQMTILRDLEKLAGWHRIAIIF | |
| | ILSGITGNLASAIFLPYRAEVGPAGSQFGLLACLFVELFQS | |
| | WPLLERPWKAFLNLSAIVLFLFICGLLPWIDNIAHIFGFLS | |
| | GLLLAFAFLPYITFGTSDKYRKRALILVSLLAFAGLFAALV | |
| | LWLYIYPINWPWIEHLTCFPFTSRFCEKYELDQVLH | |
| 17 | MSEARRDSTSSLQRKKPPWLKLDIPSAVPLTAEEPSFLQPL | >NP_071895.3 Human iRhom1 |
| | RRQAFLRSVSMPAETAHISSPHHELRRPVLQRQTSITQTIR | |
| | RGTADWFGVSKDSDSTQKWQRKSIRHCSQRYGKLKPQVLRE | |
| | LDLPSQDNVSLTSTETPPPLYVGPCQLGMQKIIDPLARGRA | |
| | FRVADDTAEGLSAPHTPVTPGAASLCSFSSSRSGFHRLPRR | |
| | RKRESVAKMSFRAAAALMKGRSVRDGTFRRAQRRSFTPASF | |
| | LEEDTTDFPDELDTSFFAREGILHEELSTYPDEVFESPSEA | |
| | ALKDWEKAPEQADLTGGALDRSELERSHLMLPLERGWRKQK | |
| | EGAAAPQPKVRLRQEVVSTAGPRRGQRIAVPVRKLFAREKR | |
| | PYGLGMVGRLTNRTYRKRIDSFVKRQIEDMDDHRPFFTYWL | |
| | TFVHSLVTILAVCIYGIAPVGFSQHETVDSVLRNRGVYENV | |
| | KYVQQENFWIGPSSEALIHLGAKFSPCMRQDPQVHSFIRSA | |
| | REREKHSACCVRNDRSGCVQTSEEECSSTLAVWVKWPIHPS | |
| | APELAGHKRQFGSVCHQDPRVCDEPSSEDPHEWPEDITKWP | |
| | ICTKNSAGNHTNHPHMDCVITGRPCCIGTKGRCEITSREYC | |
| | DFMRGYFHEEATLCSQVHCMDDVCGLLPFLNPEVPDQFYRL | |
| | WLSLFLHAGILHCLVSICFQMTVLRDLEKLAGWHRIAIIYL | |
| | LSGVTGNLASAIFLPYRAEVGPAGSQFGILACLFVELFQSW | |
| | QILARPWRAFFKLLAVVLFLFTFGLLPWIDNFAHISGFISG | |
| | LFLSFAFLPYISFGKFDLYRKRCQIIIFQVVFLGLLAGLVV | |
| | LFYVYPVRCEWCEFLTCIPFTDKFCEKYELDAQLH | |
| 18 | AQHVITCILVLRNKGVYE | Peptide sequence in iRhom2 which |
| | | corresponds to immunization |
| | | peptide 1 |
| 19 | APVGFAQHVTTQLVLRNKGVYE | Peptide sequence in iRhom2 which |
| | | corresponds to immunization |
| | | peptide 2 |
| 20 | AQHVITQLVLRNKGVYESVKYIQQENFWV | Peptide sequence in iRhom2 which |
| | | corresponds to immunization |
| | | peptide 3 |
| 21 | APVGFAQHVTTQLVLRNKGVYESVKYIQQENFWV | Peptide sequence in iRhom2 which |
| | | corresponds to immunization |
| | | peptide 4 |
| 22 | SPCIRKDGQIEQLVLRERDLERDSG | Peptide sequence in iRhom2 which |
| | | corresponds to immunization |
| | | peptide 5 |
| 23 | CIQTQRKDCSETLATFVKWQDDTGPPMDKSDLGQKRTSGAV | Peptide sequence in iRhom2 which |
| | | corresponds to immunization |
| | | peptide 6 |
| 24 | TEQARSNHTGFLHMDCEIKGRPC | Peptide sequence in iRhom2 which |
| | | corresponds to immunization |
| | | peptide 7 |
| 25 | YIYPINWPWIEHLTCF | Peptide sequence in iRhom2 which |
| | | corresponds to immunization |
| | | peptide 8 |
| 26 | LVLWLYIYPINWPWIEHLTCF | Peptide sequence in iRhom2 which |
| | | corresponds to immunization |
| | | peptide 9 |
| 27 | YIYPINWPWIEHLTCFPFTSRFCEKYELDQVLH | Peptide sequence in iRhom2 which |
| | | corresponds to immunization |
| | | peptide 10 |
| 28 | LVLWLYIYPINWPWIEHLTCFPFTSRFCEKYELDQVLH | Peptide sequence in iRhom2 which |
| | | corresponds to immunization |
| | | peptide 11 |
| 29 | SQHETVDSVLRNRGVYE | Peptide sequence in iRhom1 which |
| | | corresponds to peptide 1b |
| 30 | APVGFSQHETVDSVLRNRGVYE | Peptide sequence in iRhom1 which |
| | | corresponds to peptide 2b |
| 31 | SQHETVDSVLRNRGVYENVKYVQQENFWI | Peptide sequence in iRhom1 which |
| | | corresponds to peptide 3b |
| 32 | APVGFSQFIETVDSVLRNRGVYENVKYVQQENFWI | Peptide sequence in iRhom1 which |
| | | corresponds to peptide 4b |
| 33 | EVQLQQSGPELVKPGASVKISCKASGYTFTDYYMNWVKQSHGKSL | HC VD of anti JMD1 antibody 4H8- |
| | EWIGDINPNNGGTSYNQKFKGKATLTVDKSSNTAYMEFRSLTSED | E3. |
| | SAVYYCARRGYYGVDYWGQGTTLTVSS | |
| 34 | DYYMN | HCDR1 of anti JMD1 antibody 4H8- |
| | | E3. |
| 35 | DINPNNGGTSYNQKFKG | HCDR2 of anti JMD1 antibody 4H8- |
| | | E3. |
| 36 | RGYYGVDY | HCDR3 of anti JMD1 antibody 4H8- |
| | | E3. |
| 37 | YTFTDYYMN | HCDR1 of anti JMD1 antibody 4H8- |
| | | E3. |
| 38 | WIGDINPNNGGTSY | HCDR2 of anti JMD1 antibody 4H8- |
| | | E3. |
| 39 | RRGYYGVDY | HCDR3 of anti JMD1 antibody 4H8- |
| | | E3. |
| 40 | NIVMTQSPKSMSMSVGERVTLNCKASENVGTYVSWYQQKPEQSP | LC VD of anti JMD1 antibody 4H8- |
| | KLLIFGASNRYTGVPDRFIGSGFATDFTLTISSVQAEDLADYHC | E3. |
| | GQSYSYPYTFGGGTKLEIK | |
| 41 | KASENVGTYVS | LCDR1 of anti JMD1 antibody 4H8- |
| | | E3. |
| 42 | GASNRYT | LCDR2 of anti JMD1 antibody 4H8- |
| | | E3. |
| 43 | GQSYSYPYT | LCDR3 of anti JMD1 antibody 4H8- |
| | | E3. |
| 44 | ENVGTYVS | LCDR1 of anti JMD1 antibody 4H8- |
| | | E3. |
| 45 | LLIFGASNRYT | LCDR2 of anti JMD1 antibody 4H8- |
| | | E3. |
| 46 | GQSYSYPY | LCDR3 of anti JMD1 antibody 4H8- |
| | | E3. |

1. A protein binder that binds to human iRhom2, and inhibits and/or reduces TACE/ADAM17 activity when bound to human iRhom2, which antibody further inhibits or reduces TNFα shedding.

2. The protein binder according to claim 1, which is a monoclonal antibody, or a target-binding fragment or derivative thereof retaining target binding capacities, or an antibody mimetic.

3. The protein binder according to claim 1, wherein the inhibition or reduction of TACE/ADAM17 activity is caused by interference with iRhom2-mediated TACE/ADAM17 activation.

4. (canceled)

5. The protein binder according to claim 1, wherein the human iRhom2 to which the protein binder binds comprises
a) the amino acid sequence set forth in SEQ ID NO 16, or
b) an amino acid sequence that has at least 80% sequence identity with SEQ ID NO 16, with the proviso that said sequence maintains iRhom2 activity.

6. The protein binder according to claim 1, which binds to the juxtamembrane domain adjacent to the transmembrane domain 1 (TMD1) of human iRhom2, which juxtamembrane domain comprises amino acid residues 431-459 of SEQ ID NO 16.

7. The protein binder according to any one of the aforementioned claims, which binds to an amino acid sequence of human iRhom2 comprising
a) at least the amino acid sequence set forth in SEQ ID NO 3, or
b) an amino acid sequence that has at least 90% sequence identity with SEQ ID NO 3.

8. The protein binder according to claim 1, which binds to one or more amino acid sequences of human iRhom2 each comprising one or more amino acids within the amino acid sequence set forth in SEQ ID NO 3

9. The protein binder according to claim 1, which binds to at least one amino acid residue selected from the group consisting of A431, Q432, H433, V434, T435, T436, Q437, L438, V439, L440, R441, N442, K443, G444, V445, Y446, E447, S448, V449, K450, Y451, 1452, Q453, Q454, E455, N456, F457, W458, V459, wherein the numbering of the amino acid residues refers to the amino acid sequence set forth in SEQ ID NO 16 (human iRhom2).

10. The protein binder according to claim 1, which is not cross reactive with human iRhom1, or the juxtamembrane domain adjacent to the transmembrane domain 1 (TMD1) thereof

11. The protein binder according to claim 1, which is an antibody in at least one of the formats selected from the group consisting of: IgG, scFv, Fab, (Fab)2

12. The protein binder according to claim 1, which is an antibody having an isotype selected from the group consisting of IgG, IgM

13. The protein binder according to claim 1, which is a murine, chimerized, humanized, or human antibody.

14. (canceled)

15. (canceled)

16. The protein binder according to any one of the aforementioned claim 1, which protein binder
a) comprises a set of heavy chain/light chain complementarity determining regions (CDR) comprised in the heavy chain/light variable region sequence pair set forth in SEQ ID NOs 33 and 40
b) comprises a set of heavy chain/light chain complementarity determining regions (CDR) comprising the following sequences
HC CDR1 (SEQ ID NO 34 or 37)
HC CDR2 (SEQ ID NO 35 or 38)
HC CDR3 (SEQ ID NO 36 or 39)
LC CDR1 (SEQ ID NO 41 or 44)
LC CDR2 (SEQ ID NO 42 or 45), and
LC CDR3 (SEQ ID NO 43 or 46)
c) comprises the heavy chain/light chain complementarity determining regions (CDR) of b), with the proviso that at least one of the CDRs has up to 3 amino acid substitutions relative to the respective SEQ ID NO 34-39 or 41-46, and/or
d) comprises the heavy chain/light chain complementarity determining regions (CDR) of b) or c), with the proviso that at least one of the CDRs has a sequence identity of ≥66% to the respective SEQ ID NO 34-39 or 41-46,
wherein the CDRs are embedded in a suitable protein framework so as to be capable to bind to human iRhom2 with sufficient binding affinity and to inhibit or reduce TACE/ADAM17 activity.

17. The protein binder according to claim 1, wherein the framework is a human VH/VL framework.

18. The protein binder of claim 1, which comprises
a) the heavy chain/light chain variable domains (VD)
HC VD (SEQ ID NO 33), and
LC VD (SEQ ID NO 40)
b) the heavy chain/light chain variable domains (VD) of a), with the proviso that
the HCVD has a sequence identity of ≥80% to the respective SEQ ID NO 33, and/or
the LCDVD has a sequence identity of ≥80% to the respective SEQ ID NO 40,
c) the heavy chain/light chain variable domains (VD) of a) or b), with the proviso that at least one of the HCVD or LCVD has up to 10 amino acid substitutions relative to the respective SEQ ID NO 33 and/or 40.
said protein binder still being capable to bind to human iRhom2 with sufficient binding affinity and to inhibit or reduce TACE/ADAM17 activity.

19. The protein binder of claim 1, wherein at least one amino acid substitution is a conservative amino acid substitution.

20. The protein binder according to of claim 1, which protein binder has at least one of
target binding affinity of ≥50% to iRhom2, and measured by SPR, compared to that of the protein binder according to any one of the aforementioned claims, and/or
≥50% of the inhibiting or reducing effect on TACE/ADAM17 activity of the protein binder according to any one of the aforementioned claims.

21. A protein binder that competes for binding to iRhom2 with the protein binder according to claim 1.

22. A protein binder that binds to essentially the same, or the same, epitope on iRhom2 as the protein binder according to claim 1.

23. The protein binder according to claim 11, which is a monoclonal antibody, or a target-binding fragment or derivative thereof retaining target binding capacities, or an antibody mimetic.

24. A nucleic acid that encodes for a binding agent the protein binder according claim 1.

25. (canceled)

26. A pharmaceutical composition comprising the protein binder according to claim 1, and optionally one or more pharmaceutically acceptable excipients.

27. A combination comprising (i) the protein binder according to claim 1 and (ii) one or more therapeutically active compounds.

28. A method for treating or preventing an inflammatory condition, which method comprises administration, to a human or animal subject, of a composition comprising the protein binder according to claim 1, in a therapeutically sufficient dose.

29. A therapeutic kit of parts comprising:
a) the composition of claim 1,
b) an apparatus for administering the composition, and
c) instructions for use.


SEQUENCE LISTING (46 sequences)

SEQ ID NO: 1 - protein, 18 aa, artificial sequence
  immunization peptide
AQHVTTQLVLRNKGVYEC

SEQ ID NO: 2 - protein, 23 aa, artificial sequence
  immunization peptide
APVGFAQHVTTQLVLRNKGVYEC

SEQ ID NO: 3 - protein, 30 aa, artificial sequence
  immunization peptide
AQHVTTQLVLRNKGVYESVKYIQQENFWVC

SEQ ID NO: 4 - protein, 35 aa, artificial sequence
  immunization peptide
APVGFAQHVTTQLVLRNKGVYESVKYIQQENFWVC

SEQ ID NO: 5 - protein, 26 aa, artificial sequence
  immunization peptide
  Xaa (4)..(4): Xaa = aminobutyrate
CSPXIRKDGQIEQLVLRERDLERDSG

SEQ ID NO: 6 - protein, 41 aa, artificial sequence
  immunization peptide
  Xaa (9)..(9): Xaa = aminobutyrate
CIQTQRKDXSETLATFVKWQDDTGPPMDKSDLGQKRTSGAV

SEQ ID NO: 7 - protein, 23 aa, artificial sequence
  immunization peptide
  Xaa (16)..(16): Xaa = aminobutyrate
TEQARSNHTGFLHMDXEIKGRPC

SEQ ID NO: 8 - protein, 17 aa, artificial sequence
  immunization peptide
  Xaa (15)..(15): Xaa = aminobutyrate
YIYPINWPWIEHLTXFC

SEQ ID NO: 9 - protein, 22 aa, artificial sequence
  immunization peptide
  Xaa (20)..(20): Xaa = aminobutyrate
LVLWLYIYPINWPWIEHLTXFC

SEQ ID NO: 10 - protein, 34 aa, artificial sequence
  immunization peptide
YIYPINWPWIEHLTCFPFTSRFCEKYELDQVLHC

SEQ ID NO: 11 - protein, 39 aa, artificial sequence
  immunization peptide
LVLWLYIYPINWPWIEHLTCFPFTSRFCEKYELDQVLHC

SEQ ID NO: 12 - protein, 18 aa, artificial sequence
  immunization peptide
SQHETVDSVLRNRGVYEC

SEQ ID NO: 13 - protein, 23 aa, artificial sequence
  immunization peptide
APVGFSQHETVDSVLRNRGVYEC

SEQ ID NO: 14 - protein, 30 aa, artificial sequence
  immunization peptide
SQHETVDSVLRNRGVYENVKYVQQENFWIC

SEQ ID NO: 15 - protein, 35 aa, artificial sequence
  immunization peptide
APVGFSQHETVDSVLRNRGVYENVKYVQQENFWIC

SEQ ID NO: 16 - protein, 856 aa, Homo sapiens
MASADKNGGSVSSVSSSRLQSRKPPNLSITIPPPEKETQAPGEQDSMLPEGFQNRRLKKSQPRTWAAHTTACPPSFLPKRKNPAYLKSVSLQEPRSRWQESSEKRPGFRRQASLSQSIRKGAAQWFGVSGDWEGQRQQWQRRSLHHCSMRYGRLKASCQRDLELPSQEAPSFQGTESPKPCKMPKIVDPLARGRAFRHPEEMDRPHAPHPPLTPGVLSLTSFTSVRSGYSHLPRRKRMSVAHMSLQAAAALLKGRSVLDATGQRCRVVKRSFAFPSFLEEDVVDGADTFDSSFFSKEEMSSMPDDVFESPPLSASYFRGIPHSASPVSPDGVQIPLKEYGRAPVPGPRRGKRIASKVKHFAFDRKKRHYGLGVVGNWLNRSYRRSISSTVQRQLESFDSHRPYFTYWLTFVHVIITLLVICTYGIAPVGFAQHVTTQLVLRNKGVYESVKYIQQENFWVGPSSIDLIHLGAKFSPCIRKDGQIEQLVLRERDLERDSGCCVQNDHSGCIQTQRKDCSETLATFVKWQDDTGPPMDKSDLGQKRTSGAVCHQDPRTCEEPASSGAHIWPDDITKWPICTEQARSNHTGFLHMDCEIKGRPCCIGTKGSCEITTREYCEFMHGYFHEEATLCSQVHCLDKVCGLLPFLNPEVPDQFYRLWLSLFLHAGVVHCLVSVVFQMTILRDLEKLAGWHRIAIIFILSGITGNLASAIFLPYRAEVGPAGSQFGLLACLFVELFQSWPLLERPWKAFLNLSAIVLFLFICGLLPWIDNIAHIFGFLSGLLLAFAFLPYITFGTSDKYRKRALILVSLLAFAGLFAALVLWLYIYPINWPWIEHLTCFPFTSRFCEKYELDQVLH

SEQ ID NO: 17 - protein, 855 aa, Homo sapiens
MSEARRDSTSSLQRKKPPWLKLDIPSAVPLTAEEPSFLQPLRRQAFLRSVSMPAETAHISSPHHELRRPVLQRQTSITQTIRRGTADWFGVSKDSDSTQKWQRKSIRHCSQRYGKLKPQVLRELDLPSQDNVSLTSTETPPPLYVGPCQLGMQKIIDPLARGRAFRVADDTAEGLSAPHTPVTPGAASLCSFSSSRSGFHRLPRRRKRESVAKMSFRAAAALMKGRSVRDGTFRRAQRRSFTPASFLEEDTTDFPDELDTSFFAREGILHEELSTYPDEVFESPSEAALKDWEKAPEQADLTGGALDRSELERSHLMLPLERGWRKQKEGAAAPQPKVRLRQEVVSTAGPRRGQRIAVPVRKLFAREKRPYGLGMVGRLTNRTYRKRIDSFVKRQIEDMDDHRPFFTYWLTFVHSLVTILAVCIYGIAPVGFSQHETVDSVLRNRGVYENVKYVQQENFWIGPSSEALIHLGAKFSPCMRQDPQVHSFIRSAREREKHSACCVRNDRSGCVQTSEEECSSTLAVWVKWPIHPSAPELAGHKRQFGSVCHQDPRVCDEPSSEDPHEWPEDITKWPICTKNSAGNHTNHPHMDCVITGRPCCIGTKGRCEITSREYCDFMRGYFHEEATLCSQVHCMDDVCGLLPFLNPEVPDQFYRLWLSLFLHAGILHCLVSICFQMTVLRDLEKLAGWHRIAIIYLLSGVTGNLASAIFLPYRAEVGPAGSQFGILACLFVELFQSWQILARPWRAFFKLLAVVLFLFTFGLLPWIDNFAHISGFISGLFLSFAFLPYISFGKFDLYRKRCQIIIFQVVFLGLLAGLVVLFYVYPVRCEWCEFLTCIPFTDKFCEKYELDAQLH

SEQ ID NO: 18 - protein, 17 aa, Homo sapiens
AQHVTTQLVLRNKGVYE

SEQ ID NO: 19 - protein, 22 aa, Homo sapiens
APVGFAQHVTTQLVLRNKGVYE

SEQ ID NO: 20 - protein, 29 aa, Homo sapiens
AQHVTTQLVLRNKGVYESVKYIQQENFWV

SEQ ID NO: 21 - protein, 34 aa, Homo sapiens
APVGFAQHVTTQLVLRNKGVYESVKYIQQENFWV

SEQ ID NO: 22 - protein, 25 aa, Homo sapiens
SPCIRKDGQIEQLVLRERDLERDSG

SEQ ID NO: 23 - protein, 41 aa, Homo sapiens
CIQTQRKDCSETLATFVKWQDDTGPPMDKSDLGQKRTSGAV

SEQ ID NO: 24 - protein, 23 aa, Homo sapiens
TEQARSNHTGFLHMDCEIKGRPC

SEQ ID NO: 25 - protein, 16 aa, Homo sapiens
YIYPINWPWIEHLTCF

SEQ ID NO: 26 - protein, 21 aa, Homo sapiens
LVLWLYIYPINWPWIEHLTCF

SEQ ID NO: 27 - protein, 33 aa, Homo sapiens
YIYPINWPWIEHLTCFPFTSRFCEKYELDQVLH

SEQ ID NO: 28 - protein, 38 aa, Homo sapiens
LVLWLYIYPINWPWIEHLTCFPFTSRFCEKYELDQVLH

SEQ ID NO: 29 - protein, 17 aa, Homo sapiens
SQHETVDSVLRNRGVYE

SEQ ID NO: 30 - protein, 22 aa, Homo sapiens
APVGFSQHETVDSVLRNRGVYE

SEQ ID NO: 31 - protein, 29 aa, Homo sapiens
SQHETVDSVLRNRGVYENVKYVQQENFWI

SEQ ID NO: 32 - protein, 34 aa, Homo sapiens
APVGFSQHETVDSVLRNRGVYENVKYVQQENFWI

SEQ ID NO: 33 - protein, 117 aa, artificial sequence
  antibody sequence
EVQLQQSGPELVKPGASVKISCKASGYTFTDYYMNWVKQSHGKSLEWIGDINPNNGGTSYNQKFKGKATLTVDKSSNTAYMEFRSLTSEDSAVYYCARRGYYGVDYWGQGTTLTVSS

SEQ ID NO: 34 - protein, 5 aa, artificial sequence
  antibody sequence
DYYMN

SEQ ID NO: 35 - protein, 17 aa, artificial sequence
  antibody sequence
DINPNNGGTSYNQKFKG

SEQ ID NO: 36 - protein, 8 aa, artificial sequence
  antibody sequence
RGYYGVDY

SEQ ID NO: 37 - protein, 9 aa, artificial sequence
  antibody sequence
YTFTDYYMN

SEQ ID NO: 38 - protein, 14 aa, artificial sequence
  antibody sequence
WIGDINPNNGGTSY

SEQ ID NO: 39 - protein, 9 aa, artificial sequence
  antibody sequence
RRGYYGVDY

SEQ ID NO: 40 - protein, 107 aa, artificial sequence
  antibody sequence
NIVMTQSPKSMSMSVGERVTLNCKASENVGTYVSWYQQKPEQSPKLLIFGASNRYTGVPDRFIGSGFATDFTLTISSVQAEDLADYHCGQSYSYPYTFGGGTKLEIK

SEQ ID NO: 41 - protein, 11 aa, artificial sequence
  antibody sequence
KASENVGTYVS

SEQ ID NO: 42 - protein, 7 aa, artificial sequence
  antibody sequence
GASNRYT

SEQ ID NO: 43 - protein, 9 aa, artificial sequence
  antibody sequence
GQSYSYPYT

SEQ ID NO: 44 - protein, 8 aa, artificial sequence
  antibody sequence
ENVGTYVS

SEQ ID NO: 45 - protein, 11 aa, artificial sequence
  antibody sequence
LLIFGASNRYT

SEQ ID NO: 46 - protein, 8 aa, artificial sequence
  antibody sequence
GQSYSYPY